(19) United States
(12) Patent Application Publication
(10) Pub. No.: US 2012/0264811 A1
(43) Pub. Date: Oct. 18, 2012

(54) Elongator Proteins and Use Thereof as DNA Demethylases

(75) Inventors: Yi Zhang, Chapel Hill, NC (US); Yuki Okada, Honkomagome (JP)

(73) Assignee: The University of North Carolina at Chapel Hill, Chapel Hill, NC (US)

(21) Appl. No.: 13/499,870

(22) PCT Filed: Oct. 15, 2010
(86) PCT No.: PCT/US10/52857

§ 371 Date: Jun. 29, 2012

Related U.S. Application Data
(60) Provisional application No. 61/252,033, filed on Oct. 15, 2009.

(51) Int. Cl. C12N 9/10; C12N 5/071; G01N 21/64; C12N 15/85; A61P 35/00; A61K 31/713; C12Q 1/68

(52) U.S. Cl. 514/44 A; 435/193; 435/375; 435/6.11; 435/455

(57) ABSTRACT

The invention provides DNA demethylases comprising Elp1, Elp2, Elp3, Elp4, Elp5 and/or Elp6. The invention also provides methods of modulating gene expression, for example, for the treatment of cancer or to modify the cellular transcription program (e.g., for regenerative medicine). Also provided are methods of identifying compounds that modulate the DNA demethylase activity of the DNA demethylases of the invention.

### RELATED APPLICATION INFORMATION

This application claims the benefit of U.S. Provisional Application No. 61/252,033; filed Oct. 15, 2009, the disclosure of which is incorporated by reference herein in its entirety.

### STATEMENT OF GOVERNMENT SUPPORT

This invention was supported in part by funding provided under Grant No. GM68804 from the National Institutes of Health. The United States government has certain rights in this invention.

### FIELD OF THE INVENTION

The invention relates to DNA demethylases and methods of modulating gene expression, for example, for the treatment of cancer or to modify a cellular transcription program, as well as methods of identifying compounds that modulate DNA demethylase activity.

### BACKGROUND OF THE INVENTION

Active removal of the methyl group from 5-methyl-CpG (5mC) of DNA has been observed in at least two stages of embryogenesis. One occurs in zygotes when the paternal genome is preferentially demethylated^{1,2}. Interestingly, imprinted genes, whose expression status depends on parental origin and the methylation state of imprinting control regions (ICRs), are resistant to this wave of DNA demethylation³. Instead, this group of genes is actively demethylated at a second stage which occurs in primordial germ cells (PGCs) from E10.5 to E12.5, and results in the establishment of gender-specific methylation patterns^{4,5}. Dynamic changes in DNA methylation are not only important for early embryogenesis, but are also required for epigenetic reprogramming by somatic cell nuclear transfer (SCNT)⁶. Given the importance of active DNA demethylation in embryogenesis, reprogramming, cloning, and stem cell biology, the identification of the putative demethylase has been a major focus in the field⁷.

The first molecule claimed to possess DNA demethylase activity is the methyl-CpG binding protein Mbd2⁸. However, this protein is apparently not responsible for paternal genome demethylation as normal demethylation is still observed in Mbd2 deficient zygotes⁹. Several recent studies in plants^{10,11}, zebrafish¹², and mammalian cells^{13,14} have demonstrated that active DNA demethylation can occur through various DNA repair mechanisms (reviewed in¹⁶). However, it is not known whether any of these proteins affect paternal genome demethylation

Elp3 is a component of the elongator complex that was initially identified based on its association with an RNA polymerase II holoenzyme engaged in transcription elongation¹⁶. Subsequent studies have revealed that the elongator complex has diverse functions which include cytoplasmic kinase signaling, exocytosis, and tRNA modification¹⁷. The yeast elongator complex is composed of six subunits, Elp1-6, that include a histone acetyltransferase (HAT) Elp3¹⁸. The human elongator purified from HeLa is also composed of six subunits¹⁹.

Chinenov et al.²⁰ speculated that Elp3 has histone demethylase activity. However, this hypothesis was later disproved²¹.

### SUMMARY OF THE INVENTION

The present invention overcomes previous shortcomings in the art by identifying Elp proteins as having a central role in paternal genome demethylation.

The life cycle of mammals begins when a sperm enters an egg. Immediately after fertilization, both maternal and paternal genomes undergo dramatic reprogramming to prepare for transition from germ cell to somatic cell transcription programs²². One of the molecular events that takes place during this transition is the demethylation of the paternal genome before S-phase of the first cell cycle^{1,2}. Despite extensive efforts, the factors responsible for paternal genome DNA demethylation have not been identified⁷. As a result, there is considerable controversy in the field as to whether demethylation occurs by a passive or active mechanism^{23,24}.

To search for such factors, the inventors developed a live imaging system which allows for the methylation state of paternal DNA to be monitored. Through siRNA-mediated knockdown in zygotes, the inventors identified Elp3/KAT9, a component of the elongator complex¹⁷, to be involved in paternal DNA demethylation. The inventors demonstrate that knockdown of Elp3, as well as two additional elongator components, Elp1 and Elp4, prevented paternal genome demethylation from occurring. Importantly, injection of mRNA encoding an Elp3 radical SAM domain mutant, but not HAT domain mutant, into MII oocytes before fertilization, blocked paternal DNA demethylation, indicating that the radical SAM domain is important for the demethylation process. Consistent with this notion, injection of butylated hydroxytoluene, a radical quencher, also blocked DNA demethylation. Thus, these studies demonstrate a central function of Elp3 in paternal genome demethylation, and suggests a radical SAM initiated reaction as the mechanism driving this molecular event.

Accordingly, as a first aspect the invention provides a recombinant or isolated DNA demethylase (e.g., a mammalian demethylase) comprising Elp3.

The invention further provides a DNA demethylase comprising a complex comprising Elp3, and optionally one or more of Elp1, Elp2, Elp4, Elp5 or Elp6, in any combination.

As another aspect, the invention provides a method of demethylating DNA in a cell (e.g., a mammalian cell), the method comprising introducing a DNA demethylase according to the invention into the cell.

As a further aspect, the invention provides a method of reducing DNA demethylation in a cell (e.g., a mammalian cell), the method comprising reducing the activity of Elp1, Elp2, Elp3, Elp4, Elp5 or Elp6, or any combination thereof, in the cell. Optionally, the cell is implanted into a subject.

As yet another aspect, the invention provides a method of preventing or treating cancer in a subject (e.g., a mammalian subject) in need thereof, the method comprising administering to the subject an effective amount of one or more nucleic acids encoding a DNA demethylase of the invention.

Still further, the invention provides a method of preventing or treating cancer in a subject (e.g., a mammalian subject) in need thereof, the method comprising reducing the activity of Elp1, Elp2, Elp3, Elp4, Elp5 or Elp6, or any combination thereof in the subject.

The invention also encompasses a method of modifying a transcriptional program in a cell (e.g., a mammalian cell), the method comprising introducing Elp3 into the cell.

Further provided is a method of modifying a transcriptional program in a cell (e.g., a mammalian cell), the method comprising introducing a DNA demethylase comprising a complex comprising Elp3, and optionally one or more of Elp1, Elp2, Elp4, Elp5 or Elp6, in any combination.

As a further aspect, the invention provides a method of identifying a compound that modulates the DNA demethylase activity of Elp3 (e.g., a recombinant and/or mammalian Elp3), the method comprising:

(a) contacting the Elp3 with a DNA substrate in the presence of a test compound; and

(b) detecting the level of demethylation of the DNA substrate under conditions sufficient for DNA demethylation, wherein a change in demethylation of the DNA substrate as compared with the level of demethylation in the absence of the test compound indicates that the test compound is a modulator of the DNA demethylase activity of Elp3.

The invention also provides a method of identifying a compound that modulates the DNA demethylase activity of a complex (e.g., a recombinant and/or mammalian complex) comprising Elp1, Elp2, Elp3, Elp4, Elp5 or Elp6, or any combination thereof, the method comprising:

(a) contacting the complex with a DNA substrate in the presence of a test compound; and

(b) detecting the level of demethylation of the DNA substrate under conditions sufficient for DNA demethylation, wherein a change in demethylation of the DNA substrate as compared with the level of demethylation in the absence of the test compound indicates that the test compound is a modulator of the DNA demethylase activity of the complex.

Further provided is a method of identifying a candidate compound for the treatment of cancer, the method comprising:

(a) contacting an Elp3 (e.g., a recombinant and/or mammalian Elp3) with a DNA substrate in the presence of a test compound; and

(b) detecting the level of demethylation of the DNA substrate under conditions sufficient for DNA demethylation, wherein a change in demethylation of the DNA substrate as compared with the level of demethylation in the absence of the test compound indicates that the test compound is a candidate compound for the treatment of cancer.

As yet a further aspect, the invention provides a method of identifying a candidate compound for the treatment of cancer, the method comprising:

(a) contacting a complex (e.g., a recombinant and/or mammalian complex) comprising Elp1, Elp2, Elp3, Elp4, Elp5 or Elp6 or any combination thereof with a DNA substrate in the presence of a test compound; and

(b) detecting the level of demethylation of the DNA substrate under conditions sufficient for DNA demethylation, wherein a change in demethylation of the DNA substrate as compared with the level of demethylation in the absence of the test compound indicates that the test compound is a candidate compound for the treatment of cancer.

Another aspect of the invention provides a method of identifying a candidate compound for the modulation of gene expression in a cell, the method comprising:

(a) contacting an Elp3 (e.g., a recombinant and/or mammalian Elp3) with a DNA substrate in the presence of a test compound; and

(b) detecting the level of demethylation of the DNA substrate under conditions sufficient for DNA demethylation, wherein an increase in demethylation of the DNA substrate as compared with the level of demethylation in the absence of the test compound indicates that the test compound is a candidate compound for modulating gene expression in a cell.

The invention also provides a method of identifying a candidate compound for modulating gene expression in a cell, the method comprising:

(a) contacting a recombinant mammalian complex comprising Elp1, Elp2, Elp3, Elp4, Elp5 or Elp6 or any combination thereof with a DNA substrate in the presence of a test compound; and

(b) detecting the level of demethylation of the DNA substrate under conditions sufficient for DNA demethylation, wherein an increase in demethylation of the DNA substrate as compared with the level of demethylation in the absence of the test compound indicates that the test compound is a candidate compound for modulating gene expression in a cell.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

These and other aspects of the invention are addressed in more detail in the description of the invention set forth below.

### BRIEF DESCRIPTION OF THE DRAWINGS

FIG. 1. Gadd45b-deficiency does not affect paternal DNA demethylation. (a) Relative expression level of Gadd45 family members in mouse zygotes (A,B, and C). (b) 5mC staining of wild type and Gadd45b-deficient zygotes at pronuclear (PN) stage 4-5. Pronuclear staging and genders were determined based on criteria defined previously¹¹. 5mC-positive signal was detected using FITC-labeled secondary antibody (left column). DNAs were stained with PI (middle column). ♂: male pronucleus, ♀: female pronucleus, PB: polar body. Bar=25 μm.

FIG. 2. Construction and evaluation of a CxxC-EGFP reporter for monitoring DNA methylation state in real-time. (a) Domain/motif structure of MBD1 and MLL1 proteins. (b) Schematic representation of MBD-EGFP and CxxC-EGFP expression constructs and the expected subcellular distribution of the encoded proteins. The CMV promoter allows for expression in mammalian cells and the T7 promoter allows for in vitro generation of mRNA. An optimal polyA tail was engineered for efficient translation in zygotes. (c) Subcellular distribution of EGFP-MBD (left) and CxxC-EGFP (right) reporters in p53 knockout (normal DNA methylation, top panels) and p53/Dnmt1 double knockout (low DNA methylation, bottom panels). (d) Quantification of the results shown in (c). The data is presented as percentage of cells with nuclear dots over total transfected cells. (e) Enhanced nuclear dot-formation of CxxC probe by 5-Aza-dC-mediated DNA demethylation. NIH3T3 cells that stably express CxxC-EGFP were selected in the presence of 1 mg/ml G418. 5-Aza-dC (Sigma-Aldrich) was applied at the concentration of 5 μM for 72 hours before DAPI staining and imaging.

FIG. 3. Evaluation of CxxC-EGFP reporter in zygotes. (a) Scheme of the experimental design. (b) Representative images to illustrate the dynamics of CxxC-EGFP distribution during zygotic development by time-lapse imaging. ♂: male pronucleus, ♀: female pronucleus. Bar=25 μm.

FIG. 4. Knockdown of Elp3 prevents preferential incorporation of the CxxC-EGFP reporter into the paternal pronucleus. (a) Scheme of the experimental procedure. (b, c) Time-lapse imaging of CxxC-EGFP (left column) and H3.3-mRFP1 (middle column) at various pronucleus stages of zygotic development in the absence (b) or presence (c) of siRNA that targets Elp3. ♂: male pronucleus, ♀: female pronucleus, PB: polar body. Bar=25 μm.

FIG. 5. List of candidates with over 80% of knockdown achieved in zygotes and the distribution of the CxxC-EGFP at PN4-5 stage. (a) A list of tested candidates with over 80% of knockdown by RNAi. Knockdown efficiency was determined by RT-qPCR. (b) Representative images of CxxC-EGFP distribution at PN4-5 after RNAi. ♂: male pronucleus, ♀: female pronucleus, PB: polar body. Bar=25 μm.

FIG. 6. Knockdown of Elp3 impairs DNA demethylation in the paternal pronucleus. (a) siRNA-mediated knockdown of Elp3 resulted in increased 5mC staining in the PN5 paternal pronucleus. H3.3-mRFP1 serves as a nuclear marker. H3.3-mRFP1 signal is more intense in male pronuclei than in female pronuclei due to preferential incorporation of H3.3 into the paternal genome. ♂: male pronucleus, ♀: female pronucleus, PB: polar body. Bar=25 μm. (b) Quantification of the ratio (male/female) of 5mC intensity in Elp3 knockdown and control groups. Each symbol represents a zygote. Filled bars represent the averages ratio of each group. The statistics of the injections are presented in the table. (c) Bisulfite sequencing of Line1-5′ and ETn indicates that knockdown of Elp3 impairs paternal DNA demethylation. Open circles and closed circles represent unmethylated and methylated CpG, respectively. Each line represents an individual clone. 10 CpGs and 15 CpGs were analyzed for Line-1-5′ and ETn, respectively.

FIG. 7. Representative images of 5mC staining in PN4-5 zygotes with (lower panel) or without (upper panel) Elp3 siRNA. Paternal and maternal pronuclei are indicated by solid and dotted circles, respectively.

FIG. 8. Quantification of 5mC intensity using MetaMorph. (a) Series of Z-sectioned images were pseudocolored to identify the section which contained either male or female pronuleus (PN) with the highest 5mC intensity. In this example, Section #9 contained the female PN with the highest intensity, whereas #18 contained male PN with the highest intensity. The value was calculated as a ratio (male/female) of 5mC intensity. (b) Representative Z-stacked images of 5mC staining in zygotes with different ♂/♀values. ♂: male PN, ♀: female PN.

FIG. 9. Knockdown of the elongator complex components Elp1 and Elp4 also impairs DNA demethylation in the paternal pronucleus. (a) siRNA-mediated knockdown of Elp1 and Elp4 resulted in increased 5mC staining in the PN5 paternal pronucleus. H3.3-mRFP1 serves as a nuclear marker. ♂: male pronucleus, ♀: female pronucleus, PB: polar body. Bar=25 μm. (b) Quantification of the ratio (male/female) of 5mC intensity in Elp1, Elp4, Elp3 knockdown and control groups. Each symbol represents a zygote. Red bars represent the averages ratio of each group. The statistics of the injections are presented in the table.

FIG. 10. Mutation of the cysteine-rich radical SAM domain of Elp3 impairs paternal DNA demethylation. (a) Schematic representation of wild-type and mutant mElp3. Conserved domain (CD) of Elp3 protein sequences (SEQ ID NOs:5 and 6) from NCBI are aligned with Elp3 sequences from budding yeast (yElp3p; SEQ ID NOs:7 and 8), and mouse (mElp3; SEQ ID NOs:9 and 10). Conserved amino acid residues are underlined. (b) Overexpression of the Cys mutant, but not the wild-type or HAT mutant, blocked paternal DNA demethylation. Representative images from PN5 stage were shown. ♂: male pronucleus, ♀: female pronucleus, PB: polar body. Bar=25 μm. (c) Quantification of the ratio (male/female) of 5mC intensity in control, and Elp3 (wild-type, Cys mutant, or HAT mutant) mRNA injected groups. Each dot represents a zygote. Red bars represent the averages ratio of each group. The statistics of the injections are presented in the table.

FIG. 11. Relative expression levels of Elp family members at different zygotic stages determined by RT-qPCR. Results are normalized by 18S, and the MII expression level is set as 1.0. H1oo and MuERVL are served as controls whose expression patterns during the zygotic development are consistent with previous reports.

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

### DEFINITIONS

The following terms are used in the description herein and the appended

The singular forms “a,” “an” and “the” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed.

As used herein, the transitional phrase “consisting essentially of” is to be interpreted as encompassing the recited materials or steps “and those that do not materially affect the basic and novel characteristic(s)” of the claimed invention (e.g., DNA demethylase activity). See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

The terms “change,” “changes” and “changing” and similar terms include both reductions and increases.

The terms “modulate,” “modulates” and “modulation” include both reductions and increases.

As used herein, the terms “reduce,” “reduces,” “reduction” and similar terms mean a decrease of at least about 25%, 35%, 50%, 75%, 80%, 85%, 90%, 95%, 97% or more. In particular embodiments, the reduction results in no or essentially no (i.e., an insignificant amount, e.g., less than about 10% or even 5%) detectable activity.

As used herein, the terms “increase,” “increases,” “increasing” and similar terms indicate an elevation of at least about 25%, 50%, 75%, 100%, 150%, 200%, 300%, 400%, 500% or more.

As used herein, the term “polypeptide” encompasses both peptides and proteins, unless indicated otherwise.

As used herein, “recombinant” refers to a product formed by using recombinant technology, i.e., created utilizing genetic engineering techniques, which are well known in the art.

A “reconstituted” complex refers to a complex that is formulated from individual, recombinant components.

As used herein, “nucleic acid” encompasses both RNA and DNA, including cDNA, genomic DNA, synthetic (e.g., chemically synthesized) DNA and chimeras of RNA and DNA. The nucleic acid may be double-stranded or single-stranded. Where single-stranded, the nucleic acid may be a sense strand or an antisense strand. The nucleic acid may be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

The term “heterologous nucleic acid” is a well-known term of art and would be readily understood by one of skill in the art to be a nucleic acid that is not normally present within the host cell and/or vector into which it has been introduced. A heterologous nucleic acid can also be an additional copy of a nucleic acid that is endogenous to the cell, where the additional copy is introduced into the cell.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide.

Subjects according to the present invention include both avians and mammals. Mammalian subjects include but are not limited to humans, non-human mammals, non-human primates (e.g., monkeys, chimpanzees, baboons, etc.), dogs, cats, mice, hamsters, rats, horses, cows, pigs, rabbits, sheep and goats. Avian subjects include but are not limited to chickens, turkeys, ducks, geese, quail and pheasant, and birds kept as pets (e.g., parakeets, parrots, macaws, cockatoos, and the like). In particular embodiments, the subject is from an endangered mammalian or avian species. In particular embodiments, the subject is a laboratory animal. Human subjects include neonates, infants, juveniles, and adults.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom and/or parameter is achieved and/or there is a delay in the progression of the disease or disorder.

The terms “prevent,” “preventing” and “prevention” (and grammatical variations thereof) refer to avoidance, prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

An “effective amount,” as used herein, refers to an amount that imparts a desired effect, which is optionally a therapeutic or prophylactic effect.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The term “cancer” has its understood meaning in the art, for example, an uncontrolled or unregulated cellular proliferation that has the potential to spread to distant sites of the body (i.e., metastasize). Exemplary cancers include, but are not limited to melanoma and other skin cancers, adenocarcinoma, thymoma, lymphoma (e.g., non-Hodgkin's lymphoma, Hodgkin's lymphoma), osteosarcoma, angiosarcoma, fibrosarcoma and other sarcomas, lung cancer, liver cancer, colon cancer, leukemia, breast cancer, uterine cancer, ovarian cancer, cervical cancer, vulvar cancer, uretal cancer, bladder cancer, prostate cancer, testicular cancer and other genitourinary cancers, kidney cancer, esophageal cancer, stomach cancer and other gastrointestinal cancers, endocrine cancers, pancreatic cancer, sinus tumors, brain or central nervous system (CNS) or peripheral nervous system (PNS) tumors, malignant or benign, including gliomas and neuroblastomas and any other cancer or malignant condition now known or later identified. In representative embodiments, the invention provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In particular embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

“Cells” used in carrying out the present invention are, in general, mammalian cells or avian cells. Mammalian cells include but are not limited to human, non-human mammal, non-human primate (e.g., monkey, chimpanzee, baboon), dog, cat, mouse, hamster, rat, horse, cow, pig, rabbit, sheep and goat cells. Avian cells include but are not limited to chicken, turkey, duck, geese, quail, and pheasant cells, and cells from birds kept as pets (e.g., parakeets, parrots, macaws, cockatoos, and the like). In particular embodiments, the cell is from an endangered mammalian or avian species. In particular embodiments, the cell is from a species of laboratory animal.

As used herein, an “isolated cell” is a cell that has been removed from a subject or is derived from a cell that has been removed from a subject, and has been enriched or at least partially purified from the tissue or organ (e.g., blood, skin, bone marrow, reproductive organ) with which it is associated in its native state.

“Totipotent” as used herein, refers to a cell that has the capacity to form an entire organism.

“Pluripotent” as used herein refers to a cell that has complete differentiation versatility, e.g., the capacity to grow into any of the animal's cell types. A pluripotent cell can be self-renewing, and can remain dormant or quiescent. Unlike a totipotent cell, a pluripotent cell cannot usually form a new blastocyst or blastoderm.

“Multipotent cell” as used herein refers to a cell that has the capacity to grow into any of a subset of cell types of the corresponding animal. Unlike a pluripotent cell, a multipotent cell does not have the capacity to form all of the cell types of the corresponding animal.

As used herein, the terms “express,” “expressing,” or “expression” (or grammatical variants thereof) in reference to a gene or coding sequence can refer to transcription to produce an RNA and, optionally translation to produce a polypeptide. Thus, unless the context indicates otherwise, the terms “express,” “expressing,” “expression” and the like can refer to events at the transcriptional, post-transcriptional, translational and/or post-translational level.

As used herein, the terms “silenced” and “silencing” with respect to a DNA, gene or coding sequence refers to inhibition of transcription, for example by C or CpG methylation.

The terms “Elp1,” “Elp2,” “Elp3,” “Elp4,” “Elp5” and “Elp6” encompass naturally occurring proteins (including allelic variants, isoforms, splice variants, and the like) as well as active variants and active fragments of any of the foregoing that retain substantial DNA demethylase activity (e.g., at least about 50%, 60%, 75%, 80%, 85%, 90%, 95% or more demethylase activity as compared with the full-length native protein), and can further be partially or wholly synthetic. In some embodiments, the Elp protein is a full-length protein.

Further, the Elp1, Elp2, Elp3, Elp4, Elp5 and Elp6 proteins can be derived from any species of interest, including without limitation, mammalian species (including humans, non-human primates such as monkey, chimpanzee, baboon, dog, cat, mouse, hamster, rat, horse, cow, pig, rabbit, sheep and goat cells, insect (e.g., Drosophila), avian species (including but not limited to chicken, turkey, duck, geese, quail and pheasant), fungal species, plant species, yeast (e.g., S. pombe or S. cerevisiae), C. elegans, D. rerio (zebrafish), etc. In embodiments of the invention, the protein is derived from a mammalian species.

In particular embodiments, an active fragment or active variant of an Elp3 protein comprises the radical SAM domain (including the iron-sulfur cluster, including the cysteine-rich region located therein, and/or the glycine-rich domain similar to motif1 in several SAM-dependent methyltransferases)²⁰ and/or the histone acetyltransferase (HAT) domain.

As used herein, a “fragment” refers to a portion of the polypeptide that retains at least one biological activity normally associated with that component, e.g., DNA demethylase activity. In representative embodiments, an active fragment comprises at least about 50, 100, 150, 200, 250 or 500 consecutive amino acids of the full-length protein.

In the context of describing the biological activity of a protein in a DNA demethylase complex, the biological activity does not necessarily refer to catalytic activity, but can also refer to the activity of other components in supporting the demethylase activity of the complex, e.g., acting as a protein scaffold, ligand binding, and the like.

In particular embodiments, an active fragment or active variant of an Elp2 protein comprises one, two, three, four or more or all of the WD40 repeats and/or the RCC1 signature 2 domain²⁵.

In particular embodiments, an active fragment or active variant of an Elp protein (e.g., Elp1, Elp2, Elp3, Elp4, Elp5, Elp6) comprises a catalytic domain, a protein binding domain, a DNA binding domain, a metal binding domain, and/or a substrate binding domain.

As used herein, an “active variant” refers to an amino acid sequence that is altered by one or more amino acids and that substantially retains at least one biological activity such as DNA demethylase activity (e.g., at least about 50%, 60%, 75%, 80%, 85%, 90%, 95% or more of at least one biological activity as compared with the full-length native protein). The active variant may have “conservative” changes, wherein a substituted amino acid has similar structural or chemical properties. In particular, such changes can be guided by known similarities between amino acids in physical features such as charge density, hydrophobicity/hydrophilicity, size and configuration, so that amino acids are substituted with other amino acids having essentially the same functional properties. For example: Ala may be replaced with Val or Ser; Val may be replaced with Ala, Leu, Met, or Ile, preferably Ala or Leu; Leu may be replaced with Ala, Val or Ile, preferably Val or Ile; Gly may be replaced with Pro or Cys, preferably Pro; Pro may be replaced with Gly, Cys, Ser, or Met, preferably Gly, Cys, or Ser; Cys may be replaced with Gly, Pro, Ser, or Met, preferably Pro or Met; Met may be replaced with Pro or Cys, preferably Cys; His may be replaced with Phe or Gln, preferably Phe; Phe may be replaced with His, Tyr, or Trp, preferably His or Tyr; Tyr may be replaced with His, Phe or Trp, preferably Phe or Trp; Trp may be replaced with Phe or Tyr, preferably Tyr; Asn may be replaced with Gln or Ser, preferably Gln; Gln may be replaced with His, Lys, Glu, Asn, or Ser, preferably Asn or Ser; Ser may be replaced with Gln, Thr, Pro, Cys or Ala; Thr may be replaced with Gln or Ser, preferably Ser; Lys may be replaced with Gln or Arg; Arg may be replaced with Lys, Asp or Glu, preferably Lys or Asp; Asp may be replaced with Lys, Arg, or Glu, preferably Arg or Glu; and Glu may be replaced with Arg or Asp, preferably Asp. Once made, changes can be routinely screened to determine their effects on function.

Alternatively, an active variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, such as for example, LASERGENE™ software.

In particular embodiments, an active variant has at least about 50%, 60%, 70%, 75%, 80%, 85%, 90%, 95% 98% or more amino acid sequence similarity or identity with the amino acid sequence of a naturally occurring protein.

As is known in the art, a number of different programs can be used to identify whether a nucleic acid or polypeptide has sequence identity to a known sequence. Percent identity as used herein means that a nucleic acid or fragment thereof shares a specified percent identity to another nucleic acid, when optimally aligned (with appropriate nucleotide insertions or deletions) with the other nucleic acid (or its complementary strand). Any suitable algorithm known in the art can be employed to determine sequence identity, e.g., BLASTN. For example, to determine percent identity between two different nucleic acids, the percent identity is to be determined using the BLASTN program “BLAST 2 sequences.” This program is available for public use from the National Center for Biotechnology Information (NCBI) over the Internet²⁶. The parameters to be used are whatever combination of the following yields the highest calculated percent identity (as calculated below) with the default parameters shown in parentheses: Program—blastn Matrix—0 BLOSUM62 Reward for a match—0 or 1 (1) Penalty for a mismatch—0, −1, −2 or −3 (−2) Open gap penalty—0, 1, 2, 3, 4 or 5 (5) Extension gap penalty—0 or 1 (1) Gap x_dropoff—0 or 50 (50) Expect—10.

Percent identity or similarity when referring to polypeptides, indicates that the polypeptide in question exhibits a specified percent identity or similarity when compared with another protein or a portion thereof over the common lengths. Algorithms for determining percent identity or similarity of polypeptide sequences are known in the art, e.g., BLASTP. This program is available for public use from the National Center for Biotechnology Information (NCBI) over the Internet²⁶. Percent identity or similarity for polypeptides is typically measured using sequence analysis software. See, e.g., the Sequence Analysis Software Package of the Genetics Computer Group, University of Wisconsin Biotechnology Center, 910 University Avenue, Madison, Wis. 53705. Protein analysis software matches similar sequences using measures of homology assigned to various substitutions, deletions and other modifications. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine.

Elp1, Elp2, Elp3 and Elp4 are conserved across a wide range of species, even those that do not exhibit paternal DNA methylation. Mammalian Elp5 and Elp6 retain some regions of similarity with the yeast Elp5 and Elp6 proteins. As used herein the term “Elp3” (also known as KAT9) includes the human Elp3 protein (see, e.g., GenBank Accession No. 12654795 [amino acid} and GenBank Accession No. BC001240 [nucleotide]), as well as orthologs thereof including but not limited to orthologs from mammals (e.g., rat, mouse), Xenopus, D. rerio, C. elegans, S. pombe and S. cerevisiae (see, e.g., GenBank Accession No. 33469023 [mouse], Gen Bank Accession No. 7511380 [C. elegans], and Gen Bank Accession No. 6325171 [S. cerevisiae]) and further including active variants and active fragments of the foregoing that substantially retain at least one biological activity such as DNA demethylase catalytic activity (e.g., at least about 50%, 60%, 75%, 80%, 85%, 90%, 95% or more biological activity as compared with the native protein).

As used herein, the term “Elp1” (also known as IKAP), includes the human Elp1 protein (see, e.g., Swiss-Prot Accession No. 095163 [amino acid]; NCBI Accession No. NM_{—}003640 [nucleotide]), as well as orthologs thereof including but not limited to orthologs from mammals (e.g., rat, mouse), Xenopus, D. rerio, C. elegans, S. pombe and S. cerevisiae (see, e.g., Swiss-Prot Accession No. Q7TT37 and NCBI Accession No. NM_{—}026079 [mouse], and Swiss-Prot Accession No. Q06706 [S. cerevisiae]) and further including active variants and active fragments of the foregoing that substantially retains at least one biological activity (e.g., at least about 50%, 60%, 75%, 80%, 85%, 90%, 95% or more biological activity as compared with the native protein).

As used herein, the term “Elp2” (also known as StlP1), includes the human Elp2 protein (see, e.g., Swiss-Prot Accession No. Q61A86 [amino acid]; NCBI Accession No. NM_{—}018255 [nucleotide]), as well as orthologs thereof including but not limited to orthologs from mammals (e.g., rat, mouse), Xenopus, D. rerio, C. elegans, S. pombe and S. cerevisiae (see, e.g., Swiss-Prot Accession No. Q91 WG4 and NCBI Accession No. NM_{—}021448 [mouse] and Swiss-Prot Accession No. P42935 [S. cerevisiae]) and further including active variants and active fragments of the foregoing that substantially retains at least one biological activity (e.g., at least about 50%, 60%, 75%, 80%, 85%, 90%, 95% or more biological activity as compared with the native protein).

As used herein, the term “Elp4” includes the human Elp4 protein (see, e.g., Swiss-Prot Accession No. Q96EB1 [amino acid] and NCBI Accession No. NM_{—}019040 [nucleotide]), as well as orthologs thereof including but not limited to orthologs from mammals (e.g., rat, mouse), Xenopus, D. rerio, C. elegans, S. pombe and S. cerevisiae (see, e.g., Swiss-Prot Accession No. Q9ER73 and NCBI Accession No. NM_{—}023876 [mouse] and Swiss-Prot Accession No. Q02884 [S. cerevisiae]) and further including active variants and active fragments of the foregoing that substantially retain at least one biological activity (e.g., at least about 50%, 60%, 75%, 80%, 85%, 90%, 95% or more biological activity as compared with the native protein).

As used herein, the term “Elp5” includes the human Elp5 protein as well as Elps from other species, including but not limited to mammals (e.g., rat, mouse), Xenopus, D. rerio, C. elegans, S. pombe and S. cerevisiae and further including active variants and active fragments of the foregoing that substantially retains at least one biological activity (e.g., at least about 50%, 60%, 75%, 80%, 85%, 90%, 95% or more biological activity as compared with the native protein). The S. cerevisiae Elp5 has been identified and cloned (see, e.g., nucleotides 480990 to 481919 of chromosome VIII; NCBI Accession No. NC 001140 [nucleotide sequence] and NCBI Accession No. NP_{—}012057 [amino acid sequence]). The human (GenBank Accession No. NP_{—}056177; amino acid sequence) and mouse Elp5 (GenBank Accession No. NP_{—}061210.2; amino acid sequence) have been isolated and cloned, and share some similarity with yeast Elp5.

As used herein, the term “Elp6” includes the human Elp6 protein as well as Elps from other species, including but not limited to mammals (e.g., rat, mouse), Xenopus, D. rerio, C. elegans, S. pombe and S. cerevisiae and further including active variants and active fragments of the foregoing that substantially retains at least one biological activity (e.g., at least about 50%, 60%, 75%, 80%, 85%, 90%, 95% or more biological activity as compared with the native protein). The S. cerevisiae Elp6 has been identified and cloned (see, e.g., nucleotides 898404 to 899225 of chromosome XIII; NCBI Accession No. NC_{—}001145 [nucleotide sequence] and NCBI Accession No. NP_{—}014043 [amino acid sequence]). The human (GenBank Accession No. NP_{—}001026873) and mouse Elp6 (GenBank Accession No. NP_{—}001074850) have been isolated and cloned, and share some similarity with yeast Elp6.

As used herein, the term “methylation” refers to the addition of a methyl group to cytosine (e.g., in genomic DNA), for example to the 5 position of cytosine to produce 5-methyl cytosine. Conversely, the term “demethylation refers to the removal of a methyl group from cytosine in DNA (e.g., in genomic DNA), for example, from 5-methyl cytosine. In embodiments of the invention, the methylation/demethylation is at a CpG dinucleotide, optionally located in a CpG island. In embodiments of the invention, the methylation/demethylation is non-CpG methylation. In embodiments of the invention, the CpG is not in a CpG island.

A “delivery vector” is any molecule for the transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “delivery vector” includes both viral and nonviral (e.g., plasmid) nucleic acid molecules for introducing a nucleic acid into a cell in vitro, ex vivo and/or in vivo. A “recombinant” delivery vector refers to a viral or non-viral delivery vector that comprises one or more heterologous nucleic acids (i.e., transgenes), e.g., two, three, four, five or more heterologous nucleic acids.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, and adenovirus vectors. Non-viral vectors include plasmids, liposomes, electrically charged lipids (cytofectins), nucleic acid-protein complexes, and biopolymers. In addition to a nucleic acid of interest, a vector may also comprise one or more regulatory regions, expression control sequences, and/or selectable markers useful in selecting, measuring, and monitoring nucleic acid transfer results (e.g., delivery to specific tissues, duration of expression, etc.).

Delivery vectors may be introduced into the desired cells by methods known in the art, e.g., transfection, electroporation, microinjection, transduction, cell fusion, DEAE dextran, calcium phosphate precipitation, lipofection (lysosome fusion), or a nucleic acid vector transporter^{27,28}.

In some embodiments, a nucleic acid can be delivered to a cell in vivo by lipofection. Synthetic cationic lipids can be used to prepare liposomes for in vivo transfection of nucleic acids^{29,30,31}. The use of cationic lipids may promote encapsulation of negatively charged nucleic acids, and also promote fusion with negatively charged cell membranes³². Particularly useful lipid compounds and compositions for transfer of nucleic acids are described in International Patent Publications WO95/18863 and WO96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. In representative embodiments, transfection is directed to particular cell types in a tissue with cellular heterogeneity, such as pancreas, liver, kidney, and the brain. Lipids may be chemically coupled to other molecules for the purpose of targeting³⁰. Targeted peptides, e.g., hormones or neurotransmitters, and proteins such as antibodies, or non-peptide molecules can be coupled to liposomes chemically.

In various embodiments, other molecules can be used for facilitating delivery of a nucleic acid in vivo, such as a cationic oligopeptide (e.g., WO95/21931), peptides derived from nucleic acid binding proteins (e.g., WO96/25508) and/or a cationic polymer (e.g., WO95/21931).

It is also possible to introduce a vector in vivo as naked nucleic acid (see U.S. Pat. Nos. 5,693,622, 5,589,466 and 5,580,859). Receptor-mediated nucleic acid delivery approaches can also be used^{33,34}.

The term “transfection” or “transduction” means the uptake of exogenous or heterologous nucleic acid (RNA and/or DNA) by a cell. A cell has been “transfected” or “transduced” with an exogenous or heterologous nucleic acid when such nucleic acid has been introduced or delivered inside the cell. A cell has been “transformed” by exogenous or heterologous nucleic acid when the transfected or transduced nucleic acid imparts a phenotypic change in the cell and/or a change in an activity or function of the cell. The transforming nucleic acid can be integrated (covalently linked) into chromosomal DNA making up the genome of the cell or it can be present as a stable plasmid.

The terms “cellular transcription program” and “transcriptional program in a cell” refer to the transcriptional profile or the complement of transcripts in a cell, e.g., the transcriptome. During development, the cellular transcriptional profile is modified several times: at the time of fertilization as the cell switches from the gametic transcriptional program to the zygotic transcriptional program, and yet again when the primordial germ cells (PGCs) are formed in the embryo, as well as during cellular differentiation as the various organs and tissues form.

### DNA Demethylases.

As one aspect, the invention provides a DNA demethylase comprising, consisting essentially of, or consisting of Elp1, Elp2, Elp3, Elp4, Elp5 and/or Elp6, which polypeptide(s) can each independently be recombinant or isolated. In particular embodiments, a DNA demethylase according to the present invention catalyzes the removal of methyl groups from 5-methyl-cytosine of DNA (e.g., genomic DNA). In representative embodiments, the DNA demethylase catalyzes the removal of a methyl group from 5-methyl-CpG, optionally located in a CpG island. In embodiments of the invention, the 5-methyl-cystosine is not part of a 5-methyl-CpG dinucleotide. In embodiments of the invention, the 5-methyl-CpG is not located in a CpG island.

In representative embodiments, the DNA demethylase comprises, consists essentially of, or consists of Elp3. In embodiments of the invention, the DNA demethylase comprises a complex comprising Elp1, Elp2, Elp3, Elp4, Elp5 and/or Elp6. In representative embodiments, the DNA demethylase comprises a complex comprising Elp3. In representative embodiments, the DNA demethylase comprises a complex comprising, consisting essentially of, or consisting of (i) Elp1 and Elp3; (ii) Elp2 and Elp3; (iii) Elp3 and Elp4; (iv) Elp3 and Elp5; or (v) Elp3 and Elp6. In embodiments of the invention, the complex comprises, consists essentially of, or consists of (i) Elp1, Elp3 and Elp4; (ii) Elp1, Elp2 and Elp3; (iii) Elp2, Elp3 and Elp4; any of the foregoing may further include Elp5 and/or Elp6. In embodiments of the invention, the complex comprises, consists essentially of, or consists of Elp1, Elp2, Elp3 and Elp4, optionally further including Elp5 and/or Elp6. In particular embodiments, the complex does not comprise any one or more of Elp1, Elp2, Elp4, Elp5 and Elp6.

The complex can be an isolated native complex or a recombinant (reconstituted) complex comprising recombinant proteins.

The Elp1, Elp2, Elp3, Elp4, Elp5 and/or Elp6 or complex (isolated or recombinant) can be from any species, optionally from a mammalian species (e.g., human).

In embodiments of the invention, the DNA demethylase comprises a component (e.g., Elp1, Elp2, Elp3, Elp4, Elp5 and/or Elp6) that comprises a DNA binding domain that binds to the promoter region of a target gene (e.g., a tumor suppressor gene). In particular embodiments, the component of the DNA demethylase is a chimeric protein comprising a heterologous DNA binding domain that binds to the promoter region of a target gene (e.g., a tumor suppressor gene).

The invention also provides Elp1, Elp2, Elp3, Elp4, Elp5 and/or Elp6 or a complex as described above for use as a DNA demethylase.

In further embodiments, a recombinant DNA demethylase of the invention has enzyme activity that is substantially the same or great than the enzyme activity of the corresponding isolated native DNA demethylase (e.g., at least 70%, 80%, 90%, 95% or more).

The present invention further provides a method of producing a recombinant DNA demethylase of the present invention (as described herein), the method comprising, consisting essentially of, or consisting of providing a host cell with a heterologous nucleic acid(s) encoding the polypeptide(s) of the DNA demethylase and culturing the host cell under conditions sufficient for expression of the protein(s) and production of the recombinant DNA demethylase. In particular embodiments, the host cell comprises (a) a heterologous nucleic acid encoding Elp1; (b) a heterologous nucleic acid encoding Elp2; (c) a heterologous nucleic acid encoding Elp3; (d) a heterologous nucleic acid encoding Elp4; (e) a heterologous nucleic acid encoding Elp5; and/or (f) a heterologous nucleic acid encoding Elp6. In embodiments of the invention, the host cell comprises a heterologous nucleic acid encoding Elp3. In embodiments of the invention, the host cell comprises (a) a heterologous nucleic acid encoding Elp1; (b) a heterologous nucleic acid encoding Elp3; and (c) a heterologous nucleic acid encoding Elp4, optionally further comprising (d) a heterologous nucleic acid encoding Elp5; and/or (e) a heterologous nucleic acid encoding Elp6.

Additionally, the heterologous nucleic acid(s) encoding the component(s) of the DNA demethylase can be associated with appropriate expression control sequences, e.g., transcription/translation control signals and polyadenylation signals.

It will be appreciated that a variety of promoter/enhancer elements can be used depending on the level and tissue-specific expression desired. The promoter can be constitutive or inducible (e.g., the metallothionein promoter or a hormone inducible promoter), depending on the pattern of expression desired. The promoter can be native or foreign and can be a natural or a synthetic sequence. By foreign, it is intended that the promoter is not found in the wild-type host into which the promoter is introduced. The promoter is chosen so that it will function in the target cell(s) of interest. Moreover, specific initiation signals are generally required for efficient translation of inserted protein coding sequences. These translational control sequences, which can include the ATG initiation codon and adjacent sequences, can be of a variety of origins, both natural and synthetic. In embodiments of the invention wherein the heterologous nucleic acids encoding the components of the reconstituted complex comprise an additional sequence to be transcribed, the transcriptional units can be operatively associated with separate promoters or with a single upstream promoter and one or more downstream internal ribosome entry site (IRES) sequences (e.g., the picornavirus EMC IRES sequence).

Suitable host cells are well known in the art. See e.g., Goeddel, Gene Expression Technology Methods in Enzymology 185, Academic Press, San Diego, Calif. (1990). For example, the host cell can be a prokaryotic or eukaryotic cell. Further, it is well known that polypeptides and/or proteins can be expressed in bacterial cells such as E. coli, insect cells (e.g., the baculovirus expression system), yeast cells, plant cells or mammalian cells (e.g. human, rat, mouse, bovine, porcine, ovine, caprine, equine, feline, canine, lagomorph, simian and the like). The host cell can be a cultured cell such as a cell of a primary or immortalized cell line. The host cell can be a cell in a microorganism, animal or plant being used essentially as a bioreactor. In particular embodiments of the present invention, the host cell is any insect cell that allows for replication of well-known expression vectors. For example, the host cell can be from Spodoptera frugiperda, such as the Sf9 or Sf21 cell lines, Drosophila cell lines, or mosquito cell lines, e.g., Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O'Reilly et al., Baculovirus Expression Vectors, A Laboratory Manual, Oxford Univ. Press (1994); Samulski et al., J. Virol. 63:3822-8 (1989); Kajigaya et al., Proc. Nat'l. Acad. Sci. USA 88: 4646-50 (1991); Ruffing et al., J. Virol. 66:6922-30 (1992); Kimbauer et al., Virology 219:37-44 (1996); Zhao et al., Virology 272:382-93 (2000); and Samulski et al., U.S. Pat. No. 6,204,059.

In some embodiments, the method of producing the recombinant DNA demethylase further comprises collecting, and optionally purifying, the recombinant DNA demethylase from the cultured host cell or a culture medium from the cultured host cell. The recombinant DNA demethylase can be purified (partially or to homogeneity) according to well-known protein isolation and purification techniques to obtain the desired amount of protein and level of purity.

Accordingly, in some embodiments, purifying the recombinant DNA methylase comprises binding the expressed DNA methylase to a solid support. The solid support can be an inorganic and/or organic particulate support material comprising sand, silicas, silicates, silica gel, glass, glass beads, glass fibers, alumina, zirconia, titania, nickel, and suitable polymer materials including, but are not limited to, agarose, polystyrene, polyethylene, polyethylene glycol, polyethylene glycol grafted or covalently bonded to polystyrene (also termed PEG-polystyrene), in any suitable form known to those of skill in the art such as a particle, bead, gel or plate. The solid support can comprise a moiety, as known to those skilled in the art, that can be used to bind to the expressed recombinant DNA methylase, e.g., nickel, an antibody or an enzyme substrate (e.g., glutathione) directed to the expressed DNA methylase. Detection can be facilitated by coupling or tagging (i.e., physically linking) the desired protein or antibody directed to the protein to an appropriate detectable substance, including commercially available detectable substances.

Examples of detectable substances include, but are not limited to, various antibodies, enzymes, peptide and/or protein tags, prosthetic groups, fluorescent materials, luminescent materials, bioluminescent materials, and radioactive materials. Examples of suitable antibodies, for example antibodies against Elp1, Elp2 and Elp3 have been described previously^{25,35}. Examples of suitable enzymes include, but are not limited to, glutathione S-transferase (GST), horseradish peroxidase, alkaline phosphatase, β-galactosidase, or acetylcholinesterase. Examples of peptide and/or protein tags include, but are not limited to, a polyhistidine peptide tag, the FLAG peptide tag, maltose binding protein (MBP), thioredoxin (Trx) and calmodulin binding peptide. Examples of suitable prosthetic group complexes include, but are not limited to, streptavidin/biotin and avidin/biotin. Examples of suitable fluorescent materials include, but are not limited to, umbelliferone, fluorescein, fluorescein isothiocyanate, rhodamine, dichlorotriazinylamine fluorescein, dansyl chloride or phycoerythrin. An example of a luminescent material includes luminal. Examples of bioluminescent materials include, but are not limited to, luciferase, luciferin, and aequorin. Examples of suitable radioactive material include, but are not limited to ¹²⁵I, ¹³¹I, ³⁵I and ³H. In particular embodiments, the expressed DNA methylase comprises a purification tag (e.g., any one or more of the components can be tagged). In some embodiments, the DNA methylase of the present invention has a purity level of at least about 40%, 50%, 60%, 70%, 80%, 90%, 95%, 97%, 98%, 99% or more (w/w).

The method of producing the recombinant DNA demethylase can optionally further comprise testing the recombinant DNA demethylase that is produced for DNA demethylase activity.

In representative embodiments of the present invention, the host cell can be stably transformed with the heterologous nucleic acid(s) encoding the polypeptide(s) described above. “Stable transformation” as used herein generally refers to the integration of the heterologous nucleic acid into the genome of the host cell in contrast to “transient transformation” wherein the heterologous nucleic acid sequences introduced into the host cell do not integrate into the genome of the host cell. The term “stable transformant” can further refer to stable expression of an episome (e.g. an Epstein-Barr Virus (EBV) derived episome).

In particular embodiments, the host cell is stably transformed with a heterologous nucleic acid sequence(s) comprising nucleic acid sequence(s) encoding ELp1, Elp2, Elp3, Elp4, Elp5 and/or Elp6.

In some embodiments, the host cell comprises one or more recombinant delivery vectors comprising the heterologous nucleic acid(s) encoding the protein(s) described above. In particular embodiments, the one or more vectors comprise (i) a vector comprising a heterologous nucleic acid encoding Elp1, (ii) a separate vector comprising a heterologous nucleic acid encoding Elp2, (iii) a separate vector comprising a heterologous nucleic acid encoding Elp3; (iv) a separate vector comprising a heterologous nucleic acid encoding Elp4; (v) a separate vector comprising a heterologous nucleic acid encoding Elp5; and/or (vi) a separate vector comprising a heterologous nucleic acid encoding Elp6, in any combination.

In other embodiments, methods of producing the recombinant DNA demethylase further comprise transforming the host cell with the one or more delivery vectors. The component(s) of the DNA demethylase can each be expressed from a separate vector. Alternatively, a single vector can encode one or more of the components of the DNA demethylase.

In further embodiments, the present invention provides a host cell comprising heterologous nucleic acid(s) encoding the polypeptide(s) of the recombinant DNA demethylase. In particular embodiments, the host cell comprises (a) a heterologous nucleic acid encoding Elp1, (b) a heterologous nucleic acid encoding Elp2, (c) a heterologous nucleic acid encoding Elp3; (d) a heterologous nucleic acid encoding Elp4, (e) a heterologous nucleic acid encoding Elp5, and/or (f) a heterologous nucleic acid encoding Elp6. Suitable host cells are described above. In some embodiments, the host cell is an insect cell (e.g., an Sf9 cell) or a mammalian cell.

Further, the host cell can be stably transformed with the heterologous nucleic acid(s) encoding the polypeptide(s) of the recombinant DNA demethylase, e.g., a heterologous nucleic acid encoding Elp1, a heterologous nucleic acid encoding Elp2, a heterologous nucleic acid encoding Elp3, a heterologous nucleic acid encoding Elp4, a heterologous nucleic acid encoding Elp5, and/or a heterologous nucleic acid encoding Elp6. In some embodiments, the host cell comprises one or more recombinant delivery vectors comprising the heterologous nucleic acid(s) as described above. In further embodiments, the one or more vectors comprise a vector comprising a heterologous nucleic acid encoding (i) Elp1, (ii) a separate vector comprising a heterologous nucleic acid encoding Elp2, (iii) a separate vector comprising a heterologous nucleic acid encoding Elp3, (v) a separate vector comprising a heterologous nucleic acid encoding Elp5, (vi) a separate vector comprising a heterologous nucleic acid encoding Elp6. Suitable vectors are described herein. According to embodiments of the present invention, the vector can be a baculovirus vector.

### Methods of Modulating Gene Expression.

The DNA demethylases of the invention can be used to modulate gene expression in a cell. For example, a DNA demethylase of the invention can be delivered to a cell to increase gene expression and/or to modify the cellular transcription program. The invention can also be practiced to treat cancer. In embodiments of the invention, the increase in gene expression is selective, i.e., it is not a global or nonspecific enhancement of transcription and/or translation of cellular DNA. To illustrate, expression of one or more methylated gene(s) in the cell (e.g., a gene methylated in the promoter region) can be increased by delivering a DNA methylase of the invention to the cell, for example, expression of one or more gene(s) that are subject to partial or complete silencing due to the presence of 5-methyl-C or 5-methyl-CpG, for example due to the presence of 5-methyl C or 5-methyl-CpG in the promoter region, can be increased by delivering a DNA demethylase of the invention to the cell.

The invention also provides a method of demethylating DNA in a cell, the method comprising introducing a DNA demethylase of the invention into a cell. In particular embodiments, the method comprises introducing Elp1, Elp2, Elp3, Elp4, Elp5 and/or Elp6 into the cell, wherein the Elp protein(s) can be isolated or recombinant and can be from any species (e.g., a mammalian Elp protein such as a human Elp protein). In embodiments of the invention, the DNA demethylase comprises a complex comprising Elp1, Elp2, Elp3, Elp4, Elp5 and/or Elp6, as described in more detail herein. In representative embodiments of the foregoing methods, the cell is a mammalian cell.

The invention also provides a method of demethylating DNA, in a cell or in a cell-free system, the method comprising contacting the DNA with a DNA demethylase of the invention. In particular embodiments, the method comprises contacting the DNA with Elp1, Elp2, Elp3, Elp4, Elp5 and/or Elp6 or a complex comprising any one or more of the foregoing, wherein the Elp protein(s) can be isolated or recombinant and can be from any species (e.g., a mammalian Elp protein such as a human Elp protein).

The term “demethylating DNA in a cell” and similar terms can refer to demethylation of one or more unspecified genes and/or can refer to demethylation of one or more identified genes (e.g., a tumor suppressor gene). In embodiments of the invention, one or more methylated gene(s) in the cell can be demethylated, for example, one or more gene(s) that are subject to partial or complete silencing due to the presence of 5-methyl-C (e.g., 5-methyl-CpG) can be demethylated to increase expression thereof. In embodiments of the invention, the gene is an imprinted gene.

Similarly, the term “reducing DNA demethylation in a cell” and similar terms can refer to decreased demethylation of one or more unspecified genes and/or can refer to decreased demethylation of one or more identified genes (e.g., an oncogene). In embodiments of the invention, demethylation of one or more gene(s) in the cell can be decreased, for example, decreased demethylation of one or more gene(s) that are subject to partial or complete silencing due to the presence of 5-methyl-Cp (e.g., 5-methyl-CpG) to reduce expression thereof.

Further, in embodiments of the invention, the DNA demethylase comprises a component (e.g., Elp1, Elp2, Elp3, Elp4, Elp5 and/or Elp6) that comprises a DNA binding domain that binds to a target gene(s) (e.g., a tumor suppressor gene). As one non-limiting illustration, the DNA demethylase can comprise a component that comprises a DNA binding domain that binds to the promoter region of a target gene(s). In embodiments of the invention, the DNA binding domain binds to the methylated region(s) of the target gene, for example, a methylated region(s) in the promoter. In particular embodiments, the component of the DNA demethylase is a chimeric protein comprising a heterologous DNA binding domain that binds a target gene(s) (e.g., the promoter region of a target gene). For example, the chimeric protein can comprise a zinc finger domain fused to (or otherwise covalently bound to) a component of the DNA demethylase, where the zinc finger domain targets the DNA demethylase to the target gene(s), for example, to the promoter (e.g., binds to the target gene(s), for example, at the promoter region). This approach is similar of that used in “zinc finger nuclease-based targeting” strategies. In embodiments of the invention, the chimeric protein comprises a zinc finger domain fused to (or otherwise covalently bound to) a component of the DNA demethylase, where the zinc finger domain targets the DNA demethylase to the methylated region(s) of the target gene(s) (e.g., binds to the methylated region(s) of the gene(s)), for example, methylated regions in the promoter.

It is known in the art that methylation (e.g., promoter methylation) can result in silencing of tumor suppressor genes. Accordingly, the invention can be practiced to reduce methylation of one or more tumor suppressor genes, thereby increasing expression (e.g., transcription) of the tumor suppression gene(s). For example, the invention can be practiced to reduce methylation in the promoter region of the tumor suppressor gene.

The tumor suppressor gene can be any tumor suppressor gene now known or later identified, including tumor suppressor genes that slow down cell division, repair DNA errors and/or are involved in apoptosis. Some tumor suppressors are transcription factors or control the activity of a transcription factor. Nonlimiting examples of tumor suppressor genes include the retinoblastoma protein (pRb) gene, TP53 gene (encoding p53), Rb1 gene, PTEN gene, APC gene, CD95 gene, BRCA1 gene, BRCA2 gene, p16^{INK4a} gene, p15^{INK4b} gene, CDKN2A gene, CDKN2B gene, p16 gene, p15 gene, MLH1 gene, DCC gene, DPC4 (SMAD4) gene, MADR2/JV18 (SMAD2) gene, MEN1 gene, MTS1 gene, NF1 gene, NF2 gene, VHL gene, WT1 gene, WRN gene, MMP-8 gene, P331NG2 gene, P281NG5 gene, Lkb1 kinase gene, p471NG3 gene, Skcg-1 gene, ANX7 gene, FEZ1 gene, killin gene, TS10Q23.3 gene, WWOX gene, CAR-1 gene, Kruppel-like factor 6 (KLF6) gene, HIN-1 gene, Hippo gene, neuromedin U gene, CRIP1 gene, and ApoD gene.

Accordingly, the invention can advantageously be practiced to increase expression of one or more tumor suppressor genes, where expression of the one or more tumor suppressor genes is reduced as compared with the level of expression in a normal (e.g., healthy) cell or subject as a result of DNA methylation (e.g., promoter methylation). The invention can also be practiced with any other methylated gene (e.g., methylated in the promoter region) for which it is desirable to increase expression by demethylating the gene (e.g., demethylating the promoter region).

The DNA demethylase can be introduced into a cell by any suitable method. For example, the DNA demethylase (or nucleic acid encoding the same) can be injected into the cell. To illustrate, in the case of a recombinant DNA demethylase, nucleic acid encoding the component(s) of the DNA demethylase can be injected into the cell. In particular embodiments, the nucleic acid is mRNA, for example, for injection into a zygote.

As another approach, one or more delivery vector(s) comprising nucleic acid encoding the component(s) of the DNA demethylase can be introduced into the cell.

The invention also contemplates methods of increasing DNA methylation in a cell, the method comprising reducing the activity of a DNA demethylase (as described herein) in a cell. In embodiments, the method comprises reducing the activity of Elp1, Elp2, Elp3, Elp4, Elp5 and/or Elp6, or any combination thereof, in the cell. For example, the method can be practiced to reduce the expression of a gene that is silenced by methylation, for example, methylation of the promoter region. In particular embodiments, the invention is practiced to reduce expression of an oncogene (e.g., by increasing methylation of the promoter region of the oncogene).

Accordingly, the invention can advantageously be practiced in a cell that has increased activity of one or more oncogenes as compared with a normal (e.g., healthy) cell or subject to reduce expression by increasing the methylation state of the one or more oncogenes (e.g., the promoter region). The invention can also be practiced with any other gene for which it is desirable to reduce expression by increasing the methylation state of the gene (e.g., the promoter region).

Oncogenes are genes that when mutated or expressed at high levels promote malignancy by allowing uncontrolled proliferation and/or inhibiting apoptosis. Some oncogenes are transcription factors, kinases, growth factors or GTPases. The oncogene can be any oncogene now known or later identified. Nonlimiting examples of oncogenes include sis, ras, myc, bcr/abl, src, Her2/neu, raf, kit, myb, fyn, trk, h-tert and bcl-2.

Reducing the activity of the DNA demethylase can be achieved by any suitable method. For example, an inhibitory nucleic acid directed against Elp1, Elp2, Elp3, Elp4, Elp5 and/or Elp6 can be introduced into the cell, optionally by injecting the inhibitory nucleic acid or using a delivery vector comprising the inhibitory nucleic acid. Nonlimiting examples of inhibitory nucleic acids include siRNA, shRNA, miRNA, antisense RNA and ribozymes.

As another approach, one or more antibodies, antibody fragments, affibodies, inhibitory binding partners (or nucleic acid encoding any of the foregoing) that specifically bind to Elp1, Elp2, Elp3, Elp4, Elp5 and/or Elp6 can be introduced into the cell.

According to the foregoing methods, the cell can be a cultured or isolated cell in vitro or a cell in vivo. Isolated or cultured cells can be introduced into a subject in vivo. Further, the cell can be a gamete (e.g., an unfertilized oocyte or sperm), a germ cell (i.e., a precursor to a gamete), a zygote (having a nucleus or male and female pronuclei), a stem cell (e.g., a hematopoietic stem cell or neural stem cell), a totipotent cell, a pluripotent cell, a multipotent cell, or a differentiated cell (e.g., a terminally differentiated cell). Examples of differentiated cells include without limitation neural cells (including cells of the peripheral and central nervous systems, in particular, brain cells such as neurons and oligodendricytes), lung cells, cells of the eye (including retinal cells, retinal pigment epithelium, and corneal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, myocardial cells, bone cells (e.g., bone marrow stem cells), spleen cells, keratinocytes, fibroblasts, endothelial cells and prostate cells. The cell can further be a cancer cell, including a tumor cell. Nonlimiting examples of cancer cells include melanoma cells, adenocarcinoma cells, thymoma cells, lymphoma (e.g., non-Hodgkin's lymphoma, Hodgkin's lymphoma) cells, sarcoma cells, lung cancer cells, liver cancer cells, colon cancer cells, leukemia cells, uterine cancer cells, breast cancer cells, prostate cancer cells, ovarian cancer cells, cervical cancer cells, bladder cancer cells, kidney cancer cells, pancreatic cancer cells, brain cancer cells, esophageal cancer cells.

The invention can further be practiced for the prevention and/or treatment of cancer. In representative embodiments, the invention provides a method of preventing or treating cancer in a mammalian or avian subject at risk for or having cancer (or suspected of having cancer), the method comprising administering an effective amount of a DNA demethylase (an isolated or recombinant DNA demethylase) of the invention to the subject. In embodiments of the invention, methylation of one or more genes such as methylation of the promoter region (e.g., a tumor suppressor gene), the silencing of which is associated with cancer, is reduced and results in increased expression of the gene(s). In particular embodiments, the DNA demethylase is a recombinant DNA demethylase and the invention comprises administering an effective amount of one or more delivery vector(s) comprising nucleic acid encoding the component(s) of the DNA demethylase. In representative embodiments, the subject has reduced expression of one or more tumor suppressor genes as compared with a healthy subject that does not have cancer and/or the factor(s) putting the subject at risk for cancer. According to this embodiment, the tumor suppressor gene can have a higher degree of methylation (e.g., the promoter region has a higher degree of methylation) as compared with the level of methylation of the tumor suppressor gene in a healthy subject that does not have cancer and/or the factor(s) putting the subject at risk for cancer.

As a further aspect, the invention provides a method of preventing or treating cancer in a mammalian or avian subject at risk for or having cancer (or suspected of having cancer), the method comprising reducing the activity of a DNA demethylase in a subject. In embodiments of the invention, methylation of one or more genes (e.g., an oncogene) associated with cancer is increased (e.g., in the promoter region) and results in reduced expression of the gene(s). In representative embodiments, the method comprises reducing the activity of Elp1, Elp1, Elp3, Elp4, Elp5 and/or Elp6 in the subject. According to embodiments of the invention, the subject has elevated expression or activity of an oncogene as compared with a healthy subject that does not have cancer and/or the factor(s) putting the subject at risk for cancer. According to this embodiment, the oncogene can have a reduced level of methylation (e.g., in the promoter region) as compared with the level of methylation of an oncogene in a normal (healthy) cell.

DNA methylation-mediated gene silencing is known to play a role in other disorders, such as neuronal disease. For example, DNA methylation-mediated silencing of the SMN2 gene correlates with the severity of spinal muscular atrophy (SMA), a common neuromuscular disorder. DNA demethylation has also been associated with silencing of the neurotensin/neuromedin N gene. Other examples include certain skin disorders (including skin tumors and autoimmune-related skin disorders; Li et al., (2009) J. Dermatol. Sci 54:143-9), immune senescence (including increased inflammation and autoimmune responses seen in aging; Yung et al., (2008) Autoimmunity 41:329-35; Grolleau-Julius et al., (2010) Clin Rev. Allergy Immunol. 39:42-50), beta cell dysfunction associated with intrauterine growth retardation and the development of diabetes (Woo et al., (2008) Cell Metab. 8:5-7), and Prader-Willi and Angelman syndromes (Gurrieri et al., (2009) Endocr. Dev. 14:20-8). Accordingly, the invention encompasses methods for the prevention and/or treatment of any disorder associated with DNA methylation-mediated gene silencing (e.g., a neuronal disease or other disorders as described above). In representative embodiments, the invention provides a method of preventing or treating a disorder associated with DNA methylation-mediated gene silencing in a mammalian or avian subject at risk for or having the disorder (or suspected of having the disorder), the method comprising administering an effective amount of a DNA demethylase (an isolated or recombinant DNA demethylase) of the invention to the subject. In embodiments of the invention, methylation of one or more genes, the silencing of which is associated with the disorder, is reduced and results in increased expression of the gene(s). In particular embodiments, the DNA demethylase is a recombinant DNA demethylase and the invention comprises administering an effective amount of one or more delivery vector(s) comprising nucleic acid encoding the component(s) of the DNA demethylase. In representative embodiments, the subject has reduced expression of one or more genes as compared with a healthy subject that does not have the disorder and/or the factor(s) putting the subject at risk for the disorder. According to this embodiment, the gene can have a higher degree of methylation (e.g., the promoter region has a higher degree of methylation) as compared with the level of methylation of the gene in a healthy subject that does not have the disorder and/or the factor(s) putting the subject at risk for the disorder.

Further, the invention provides a method of preventing or treating a disorder associated with over-expression of one or more genes, where the gene(s) is subject to regulation (e.g., silencing) by methylation (e.g., the promoter region) in a mammalian or avian subject at risk for or having the disorder (or suspected of having the disorder), the method comprising reducing the activity of a DNA demethylase in a subject. In embodiments of the invention, methylation of one or more genes associated with the disorder is increased and results in reduced expression of the gene(s). In representative embodiments, the method comprises reducing the activity of Elp1, Elp2, Elp3, Elp4, Elp5 and/or Elp6 in the subject. According to embodiments of the invention, the subject has elevated expression of one or more genes as compared with a healthy subject that does not have the disorder and/or the factor(s) putting the subject at risk for the disorder. According to this embodiment, the gene(s) can have a reduced level of methylation (e.g., in the promoter region) as compared with the level of methylation of the gene(s) in a healthy subject that does not have the disorder and/or the factor(s) putting the subject at risk for the disorder.

A reduction in the activity of the DNA demethylase or the Elp protein(s) can be achieved by any suitable method. For example, an effective amount of an inhibitory nucleic acid directed against Elp1, Elp2, Elp3, Elp4, Elp5 and/or Elp6 can be administered to the subject, for example, by administering a delivery vector comprising the inhibitory nucleic acid. Nonlimiting examples of inhibitory nucleic acids include siRNA, shRNA, miRNA, antisense RNA and ribozymes.

As another approach, an effective amount of one or more antibodies, antibody fragments, affibodies or inhibitory binding partners (or nucleic acid encoding any of the foregoing) that specifically bind to the DNA demethylase (e.g., bind to Elp1, Elp2, Elp3 and/or Elp4) can be administered to the subject.

Suitable subjects include both avians and mammals (each as defined herein). Optionally, the subject is “in need of” the methods of the present invention, e.g., because the subject has (or is suspected of having) or is believed at risk for cancer.

At risk individuals can be identified using methods known in the art, for example, by family history, genetic analysis, lifestyle factors, co-morbidities and/or the onset of early symptoms associated with the disease.

Ribozymes are RNA-protein complexes that cleave nucleic acids in a site-specific fashion. Ribozymes have specific catalytic domains that possess endonuclease activity^{36,3738}. For example, a large number of ribozymes accelerate phosphoester transfer reactions with a high degree of specificity, often cleaving only one of several phosphoesters in an oligonucleotide substrate^{39,40}. This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids⁴¹. For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of nucleic acid expression may be particularly suited to therapeutic applications^{42,43,44}.

MicroRNAs (miRNA) are RNA molecules, generally 21-23 nucleotides long, that can down-regulate gene expression by hybridizing to miRNA. Over-expression or diminution of a particular miRNA can be used to treat a dysfunction and has been shown to be effective in a number of disease states and animal models of disease⁴⁵. Mature miRNAs are produced from a primary transcript (pri-miRNA) that is processed into a short stem-loop structure (a pre-miRNA) that then forms the final miRNA product.

The term “antisense oligonucleotide” (including “antisense RNA”) as used herein, refers to a nucleic acid that is complementary to and specifically hybridizes to a specified DNA or RNA sequence. Antisense oligonucleotides and nucleic acids that encode the same can be made in accordance with conventional techniques. See, e.g., U.S. Pat. No. 5,023,243 to Tullis; U.S. Pat. No. 5,149,797 to Pederson et al.

Those skilled in the art will appreciate that it is not necessary that the antisense oligonucleotide be fully complementary to the target sequence as long as the degree of sequence similarity is sufficient for the antisense nucleotide sequence to specifically hybridize to its target (as defined above) and reduces production of the protein product (e.g., by at least about 30%, 40%, 50%, 60%, 70%, 80%, 90%, 95% or more).

To determine the specificity of hybridization, hybridization of such oligonucleotides to target sequences can be carried out under conditions of reduced stringency, medium stringency or even stringent conditions. Exemplary conditions for reduced, medium and stringent hybridization are as follows: (e.g., conditions represented by a wash stringency of 35-40% Formamide with 5×Denhardt's solution, 0.5% SDS and 1×SSPE at 37° C.; conditions represented by a wash stringency of 40-45% Formamide with 5×Denhardt's solution, 0.5% SDS, and 1×SSPE at 42° C.; and conditions represented by a wash stringency of 50% Formamide with 5×Denhardt's solution, 0.5% SDS and 1×SSPE at 42° C., respectively). See, e.g., Sambrook et al., Molecular Cloning, A Laboratory Manual (2d Ed. 1989) (Cold Spring Harbor Laboratory).

Alternatively stated, in particular embodiments, the antisense oligonucleotide has at least about 60%, 70%, 80%, 90%, 95%, 97%, 98% or higher sequence similarity with the complement of the target sequence and reduce production of the protein product (as defined above). In some embodiments, the antisense sequence contains 1, 2, 3, 4, 5, 6, 7, 8, 9 or 10 mismatches as compared with the target sequence.

Methods of determining percent identity of nucleic acid sequences are described in more detail elsewhere herein.

The length of the antisense oligonucleotide is not critical as long as it specifically hybridizes to the intended target and reduces production of the protein product and can be determined in accordance with routine procedures. In general, the antisense oligonucleotide is at least about eight, ten or twelve or fifteen nucleotides in length and/or less than about 20, 30, 40, 50, 60, 70, 80, 100 or 150 nucleotides in length.

An antisense oligonucleotide can be constructed using chemical synthesis and enzymatic ligation reactions by procedures known in the art. For example, an antisense oligonucleotide can be chemically synthesized using naturally occurring nucleotides or various modified nucleotides designed to increase the biological stability of the molecules and/or to increase the physical stability of the duplex formed between the antisense and sense nucleotide sequences, e.g., phosphorothioate derivatives and acridine substituted nucleotides can be used.

Examples of modified nucleotides which can be used to generate the antisense oligonucleotide include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine.

The antisense oligonucleotides can further include nucleotide sequences wherein at least one, or all, of the internucleotide bridging phosphate residues are modified phosphates, such as methyl phosphonates, methyl phosphonothioates, phosphoromorpholidates, phosphoropiperazidates and phosphoramidates. For example, every other one of the internucleotide bridging phosphate residues can be modified as described.

As another non-limiting example, one or all of the nucleotides in the oligonucleotide can contain a 2′ loweralkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides can be modified as described. See also, Furdon et al., (1989) Nucleic Acids Res. 17, 9193-9204; Agrawal et al., (1990) Proc. Natl. Acad. Sci. USA 87, 1401-1405; Baker et al., (1990) Nucleic Acids Res. 18, 3537-3543; Sproat et al., (1989) Nucleic Acids Res. 17, 3373-3386; Walder and Walder, (1988) Proc. Natl. Acad. Sci. USA 85, 5011-5015.

The antisense oligonucleotide can be chemically modified (e.g., at the 3′ and/or 5′ end) to be covalently conjugated to another molecule. To illustrate, the antisense oligonucleotide can be conjugated to a molecule that facilitates delivery to a cell of interest, enhances absorption by the nasal mucosa (e.g, by conjugation to a lipophilic moiety such as a fatty acid), provides a detectable marker, increases the bioavailability of the oligonucleotide, increases the stability of the oligonucleotide, improves the formulation or pharmacokinetic characteristics, and the like. Examples of conjugated molecules include but are not limited to cholesterol, lipids, polyamines, polyamides, polyesters, intercalators, reporter molecules, biotin, dyes, polyethylene glycol, human serum albumin, an enzyme, an antibody or antibody fragment, or a ligand for a cellular receptor.

Other modifications to nucleic acids to improve the stability, nuclease-resistance, bioavailability, formulation characteristics and/or pharmacokinetic properties are known in the art.

RNA interference (RNAi) is another useful approach for reducing production of a protein product (e.g., shRNA or siRNA). RNAi is a mechanism of post-transcriptional gene silencing in which double-stranded RNA (dsRNA) corresponding to a target sequence of interest is introduced into a cell or an organism, resulting in degradation of the corresponding mRNA. The mechanism by which RNAi achieves gene silencing has been reviewed in Sharp et al, (2001) Genes Dev 15: 485-490; and Hammond et al., (2001) Nature Rev Gen 2:110-119). The RNAi effect persists for multiple cell divisions before gene expression is regained. RNAi is therefore a powerful method for making targeted knockouts or “knockdowns” at the RNA level. RNAi has proven successful in human cells, including human embryonic kidney and HeLa cells (see, e.g., Elbashir et al., Nature (2001) 411:494-8).

Initial attempts to use RNAi in mammalian cells resulted in antiviral defense mechanisms involving PKR in response to the dsRNA molecules (see, e.g., Gil et al. (2000) Apoptosis 5:107). It has since been demonstrated that short synthetic dsRNA of about 21 nucleotides, known as “short interfering RNAs” (siRNA) can mediate silencing in mammalian cells without triggering the antiviral response (see, e.g., Elbashir et al., Nature (2001) 411:494-8; Caplen et al., (2001) Proc. Nat. Acad. Sci. 98:9742).

The RNAi molecule (including an siRNA molecule) can be a short hairpin RNA (shRNA; see Paddison et al., (2002), PNAS USA 99:1443-1448), which is believed to be processed in the cell by the action of the RNase III like enzyme Dicer into 20-25mer siRNA molecules. The shRNAs generally have a stem-loop structure in which two inverted repeat sequences are separated by a short spacer sequence that loops out. There have been reports of shRNAs with loops ranging from 3 to 23 nucleotides in length. The loop sequence is generally not critical. Exemplary loop sequences include the following motifs: AUG, CCC, UUCG, CCACC, CTCGAG, AAGCUU, CCACACC and UUCAAGAGA.

The RNAi can further comprise a circular molecule comprising sense and antisense regions with two loop regions on either side to form a “dumbbell” shaped structure upon dsRNA formation between the sense and antisense regions. This molecule can be processed in vitro or in vivo to release the dsRNA portion, e.g., a siRNA.

International patent publication WO 01/77350 describes a vector for bi-directional transcription to generate both sense and antisense transcripts of a heterologous sequence in a eukaryotic cell. This technique can be employed to produce RNAi for use according to the invention.

Shinagawa et al. (2003) Genes & Dev. 17:1340 reported a method of expressing long dsRNAs from a CMV promoter (a pol II promoter), which method is also applicable to tissue specific pol II promoters. Likewise, the approach of Xia et al., (2002) Nature Biotech. 20:1006, avoids poly(A) tailing and can be used in connection with tissue-specific promoters.

Methods of generating RNAi include chemical synthesis, in vitro transcription, digestion of long dsRNA by Dicer (in vitro or in vivo), expression in vivo from a delivery vector, and expression in vivo from a PCR-derived RNAi expression cassette (see, e.g., TechNotes 10(3) “Five Ways to Produce siRNAs,” from Ambion, Inc., Austin Tex.; available at www.ambion.com).

Guidelines for designing siRNA molecules are available (see e.g., literature from Ambion, Inc., Austin Tex.; available at www.ambion.com). In particular embodiments, the siRNA sequence has about 30-50% G/C content. Further, long stretches of greater than four T or A residues are generally avoided if RNA polymerase III is used to transcribe the RNA. Online siRNA target finders are available, e.g., from Ambion, Inc. (www.ambion.com), through the Whitehead Institute of Biomedical Research (www.jura.wi.mit.edu) or from Dharmacon Research, Inc. (www.dharmacon.com/).

The antisense region of the RNAi molecule can be completely complementary to the target sequence, but need not be as long as it specifically hybridizes to the target sequence and reduces production of the protein product (e.g., by at least about 30%, 40%, 50%, 60%, 70%, 80%, 90%, 95% or more). In some embodiments, hybridization of such oligonucleotides to target sequences can be carried out under conditions of reduced stringency, medium stringency or even stringent conditions, as defined above.

In other embodiments, the antisense region of the RNAi has at least about 60%, 70%, 80%, 90%, 95%, 97%, 98% or higher sequence identity with the complement of the target sequence and reduces production of the protein product (e.g., by at least about 30%, 40%, 50%, 60%, 70%, 80%, 90%, 95% or more). In some embodiments, the antisense region contains 1, 2, 3, 4, 5, 6, 7, 8, 9 or 10 mismatches as compared with the target sequence. Mismatches are generally tolerated better at the ends of the dsRNA than in the center portion.

In particular embodiments, the RNAi is formed by intermolecular complexing between two separate sense and antisense molecules. The RNAi comprises a ds region formed by the intermolecular basepairing between the two separate strands. In other embodiments, the RNAi comprises a ds region formed by intramolecular basepairing within a single nucleic acid molecule comprising both sense and antisense regions, typically as an inverted repeat (e.g., a shRNA or other stem loop structure, or a circular RNAi molecule). The RNAi can further comprise a spacer region between the sense and antisense regions.

The RNAi molecule can contain modified sugars, nucleotides, backbone linkages and other modifications as described above for antisense oligonucleotides.

Generally, RNAi molecules are highly selective. If desired, those skilled in the art can readily eliminate candidate RNAi that are likely to interfere with expression of nucleic acids other than the target by searching relevant databases to identify RNAi sequences that do not have substantial sequence homology with other known sequences, for example, using BLAST (available at www.ncbi.nlm.nih.gov/BLAST).

Kits for the production of RNAi are commercially available, e.g., from New England Biolabs, Inc. and Ambion, Inc.

The term “antibody” or “antibodies” as used herein refers to all types of immunoglobulins, including IgG, IgM, IgA, IgD, and IgE, as well as antibodies of any class and subclass and further encompasses antibody fragments that bind to the desired epitope/antigen. The antibody can be monoclonal or polyclonal and can be of any species of origin, including (for example) mouse, rat, rabbit, horse, goat, sheep or human, or can be a chimeric antibody, humanized, primatized or human antibody. See, e.g., Walker et al., Molec. Immunol. 26, 403-11 (1989). The antibodies can be recombinant monoclonal antibodies, for example, produced according to the methods disclosed in U.S. Pat. No. 4,474,893 or U.S. Pat. No. 4,816,567. The antibodies can also be chemically constructed, for example, according to the method disclosed in U.S. Pat. No. 4,676,980.

Antibody fragments included within the scope of the present invention include, for example, Fab, Fab′, F(ab′)2, single-chain Fv (scFv), disulfide-linked Fv, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254, 1275-1281).

The antibody can further be a diabody, linear antibody, single domain antibody, anti-idiotypic antibody, intrabody, or multispecific antibody formed from antibody fragments (e.g., may be a bispecific antibody).

Polyclonal antibodies can be produced by immunizing a suitable animal (e.g., rabbit, goat, etc.) with an antigen, collecting immune serum from the animal, and optionally separating the polyclonal antibodies from the immune serum, in accordance with known procedures.

Monoclonal antibodies can be produced in a hybridoma cell line according to the technique of Kohler and Milstein, (1975) Nature 265, 495-97. For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. coli by recombinant techniques known to those skilled in the art. See, e.g., W. Huse, (1989) Science 246, 1275-81.

Antibodies specific to a target polypeptide can also be obtained by phage display techniques known in the art.

Various immunoassays can be used for screening to identify antibodies having the desired specificity. Numerous protocols for competitive binding or immunoradiometric assays using either polyclonal or monoclonal antibodies with established specificity are well known in the art. Such immunoassays typically involve the measurement of complex formation between an antigen and its specific antibody (e.g., antigen/antibody complex formation). A two-site, monoclonal-based immunoassay utilizing monoclonal antibodies reactive to two non-interfering epitopes can be used as well as a competitive binding assay.

Affibodies are small, stable high affinity protein molecules that are engineered to specifically bind to a target. One example of an affibody protein scaffold is based on one of the domains of protein A. Unique binding properties can be achieved by randomization of a 13 amino acid stretch located in two alpha-helices that mediate protein A binding. This affibody structure has further been modified by incorporation of other amino acids. Affibodies having a desired specificity can be routinely identified from affibody libraries containing large numbers of molecules.

The invention can also be practiced to modify or “reset” the transcriptional program in a cell, which is relevant, for example, in the field of regenerative medicine or cloning of non-human mammals and avians (e.g., an endangered species or a domestic pet). For example, the efficiency of reprogramming can be increased and/or the time for reprogramming reduced by activating factors involved in the reprogramming process such as Oct4, Nanog, and the like. Reprogrammed cells produced according to this aspect of the invention can be administered to a subject to regenerate an organ or tissue (e.g., the islet cells of the pancreas, neural cells in the case of neural disorders such as Parkinson's or Alzheimer's or retinal or corneal cells for the treatment of eye disorders, blood vessels or blood vessel substitutes, cardiac valves, cardiac tissue, liver, blood cell substitutes, cartilage tissue, skeletal muscle, dermal implants, bone grafts, gum grafts and other tissues for periodontal applications, or any tissue lost or injured due to trauma or disease) or can be used in vitro to grow an organ or tissue for transplantation. In some embodiments, a cell is removed from a subject (autologous) or from an allogeneic donor, reprogrammed according to the present invention and then administered to the subject (optionally, after culturing in vitro to expand the number of cells and/or to modulate the differentiation state of the cell) or used to grow an organ or tissue in vitro, which is then transplanted into the subject. Methods of tissue engineering for regenerative medicine are known in the art, see, e.g., Methods of Tissue Engineering (Atala and Lanza, Eds., 2002), Academic Press, New York.

To illustrate, as one aspect the invention provides a method of modifying a transcriptional program in a mammalian cell, the method comprising introducing a DNA demethylase of the invention into the cell, which can be an isolated or recombinant DNA demethylase. In embodiments of the invention, the methylation state of one or more genes associated with the differentiation state of the cell is reduced resulting in increased expression of the one or more genes.

The cell can be a cultured or isolated cell in vitro or a cell in vivo. Cultured or isolated cells can be introduced into a subject in vivo. Further, the cell can be a gamete (e.g., an unfertilized oocyte or sperm), a germ cell (i.e., a precursor to a gamete), a zygote (e.g., having a nucleus or male and female pronuclei), a stem cell (e.g., a hematopoietic stem cell or neural stem cell), a totipotent cell, a pluripotent cell, a multipotent cell, or a differentiated cell (e.g., a terminally differentiated cell). Examples of differentiated cells include with out limitation neural cells (including cells of the peripheral and central nervous systems, in particular, brain cells such as neurons and oligodendricytes), lung cells, cells of the eye (including retinal cells, retinal pigment epithelium, and corneal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, myocardial cells, bone cells (e.g., bone marrow stem cells), spleen cells, keratinocytes, fibroblasts, endothelial cells and prostate cells.

In embodiments of the invention, the cell is a differentiated cell (e.g., terminally differentiated cell) and the invention is practiced to de-differentiate the cell and/or its progeny, for example, to return the cell to the multipotent state, a pluripotent state, or a totipotent state.

Methods of determining the differentiation or de-differentiation state and/or potency of cells are known in the art, e.g., by assessing markers (e.g., cell-surface markers), patterns of gene expression, differentiation potential, and the like. According to particular embodiments of the invention, the method further comprises determining the differentiation or de-differentiation state and/or potency of the cell and/or its progeny, for example, by determining the presence or absence of one or more markers (e.g., cell-surface marker), by evaluating the expression of one or more genes (e.g., lineage specific or cell-type specific genes), and/or evaluating the differentiation potential of the cell and/or its progeny in vitro or in vivo. For example, alkaline phosphatase, cytokeratin, vimentin, laminin, and/or c-kit may be suitable for identifying totipotent cells.

With respect to cloning, any method known in the art can be used to form a new blastocyst or organism. For example, the nucleus of a totipotent reprogrammed cell can be used in somatic cell nuclear transfer according to known protocols. Alternatively, the totipotent cell can be stimulated (e.g., electrical stimulation) to form a new blastocyst or embryo, also according to methods known in the art.

The DNA demethylase can be introduced into a cell by any suitable method. For example, the DNA demethylase (or nucleic acid encoding the same) can be injected into the cell. To illustrate, in the case of a recombinant DNA demethylase, nucleic acid encoding the component(s) of the DNA demethylase can be injected into the cell. In particular embodiments, the nucleic acid is mRNA, for example, for injection into a zygote.

As another approach, one or more delivery vector(s) comprising nucleic acid encoding the component(s) of the DNA demethylase can be introduced into the cell.

Optionally, the methods of the invention further comprise determining the methylation state of the DNA and/or the expression of a gene the expression of which is modulated by methylation (e.g., promoter methylation). Those skilled in the art will appreciate that determining the methylation state of DNA can involve directly measuring methylation or demethylation, and further can be determined on DNA as a whole, on a particular DNA fraction or with respect to one or more particular genes. Methods of measuring gene expression are known in the art, e.g., by measuring mRNA levels, transcription rates, protein levels and/or protein activity (optionally by detecting an amount and/or activity of a reporter protein).

Optionally, the invention can further comprise implanting a cell (e.g., a germ cell, an unfertilized oocyte, a zygote, a stem cell, a progenitor cell, or any other cell as described herein), treated according to the present invention into a subject. In representative embodiments, the cell is autologous to the host. In embodiments, the cell is allogeneic to the host.

### Screening Methods.

The present invention further provides methods of identifying a compound that modulates the DNA demethylase activity of a DNA demethylase of the invention. Any suitable assay for detecting or determining DNA demethylase activity can be used to identify compounds that modulate DNA demethylase activity.

In particular embodiments, the invention provides a method of identifying a compound that modulates the DNA demethylase activity of the DNA demethylase, the method comprising: (a) contacting a DNA demethylase of the invention with a DNA substrate in the presence of a test compound; and (b) detecting the level of demethylation of the DNA substrate under conditions sufficient for DNA demethylation, wherein a change in demethylation of the DNA substrate as compared with the level of demethylation in the absence of the test compound indicates that the test compound is a modulator of the DNA demethylase activity of the DNA demethylase. In particular embodiments, the DNA demethylase comprises, consists essentially of, or consists of Elp1, Elp2, Elp3, Elp4, Elp5 and/or Elp6 or is a complex comprising, consisting essentially of, or consisting of Elp1, Elp2, Elp3, Elp4, Elp5 and/or Elp6. The DNA demethylase can be isolated or recombinant.

In embodiments of the invention, a reduction in demethylation as compared with the level of demethylation in the absence of the test compound indicates that the test compound is an inhibitor of the DNA demethylase activity of the DNA demethylase.

In embodiments of the invention, an increase in demethylation as compared with the level of DNA demethylation in the absence of the test compound indicates that the test compound is an activator of the DNA demethylase activity of the DNA demethylase.

As a further aspect, the invention provides methods of identifying a candidate compound for the modulation of gene expression in a cell (e.g., for modifying the cellular transcription program) by identifying a compound that modulates the activity of a DNA demethylase of the invention. In representative embodiments, the invention provides a method of identifying a candidate compound for the modulation of gene expression in a cell, the method comprising: (a) contacting a DNA demethylase according to the invention with a DNA substrate in the presence of a test compound; and (b) detecting the level of demethylation of the DNA substrate under conditions sufficient for DNA demethylation, wherein a change in demethylation of the DNA substrate as compared with the level of demethylation in the absence of the test compound indicates that the test compound is a candidate compound for modulating gene expression in a cell. In particular embodiments, the DNA demethylase comprises, consists essentially of, or consists of Elp1, Elp2, Elp3, Elp4, Elp5 and/or Elp6 or is a complex comprising, consisting essentially of, or consisting of Elp1, Elp2, Elp3, Elp4, Elp5 and/or Elp6.

In embodiments of the invention, a reduction in demethylation as compared with the level of demethylation in the absence of the test compound indicates that the test compound is a candidate compound for inhibiting the activity of the DNA demethylase in modulating gene expression in the cell.

In embodiments of the invention, an increase in demethylation as compared with the level of demethylation in the absence of the test compound indicates that the test compound is an activator of the DNA demethylase in modulating gene expression in the cell.

Silencing of tumor suppressor genes by DNA demethylation or activation of oncogenes has been associated with cancer, indicating that drugs that can modulate DNA methylation are good candidates for cancer treatment. Accordingly, the invention also provides a method of identifying a candidate compound for treating cancer, the method comprising identifying a compound that modulates the DNA demethylase activity of a DNA demethylase of the invention.

In representative embodiments, the invention provides a method of identifying a candidate compound for the treatment of cancer, the method comprising: (a) contacting a DNA demethylase of the invention with a DNA substrate in the presence of a test compound; and (b) detecting the level of demethylation of the DNA substrate under conditions sufficient for DNA demethylation, wherein a change in demethylation of the DNA substrate as compared with the level of demethylation in the absence of the test compound indicates that the test compound is a candidate compound for the treatment of cancer. In particular embodiments, the DNA demethylase comprises, consists essentially of, or consists of Elp1, Elp2, Elp3, Elp4, Elp5 and/or Elp6 or is a complex comprising, consisting essentially of, or consisting of Elp1, Elp2, Elp3, Elp4, Elp5 and/or Elp6.

In embodiments of the invention, the DNA substrate comprises an oncogene (e.g., an activated oncogene) or any other gene that promotes cancer or tumor formation, wherein a reduction in demethylation (e.g., in the promoter region) of the oncogene or any other gene that promotes cancer or tumor formation indicates that the test compound is a candidate compound for the treatment of cancer.

In embodiments of the invention, the DNA substrate comprises a tumor suppressor gene (e.g., a silenced tumor suppressor gene, e.g., by promoter methylation) or any other gene that inhibits cancer or tumor formation, wherein an increase in demethylation (e.g., in the promoter region) of the tumor suppressor gene or any other gene that inhibits cancer or tumor formation indicates that the test compound is a candidate compound for the treatment of cancer.

Exemplary cancers are described elsewhere herein.

The DNA substrate can be a methylated DNA substrate or a nonmethylated DNA substrate.

According to the present invention, “detecting the level of demethylation” may be performed by any method known in the art. In particular embodiments, the level of DNA methylation is detected and the level of demethylation determined therefrom. Methylated or nonmethylated DNA can be detected by any method known in the art, for example, by using an antibody specific to 5-methyl-C (e.g., 5-methyl CpG) or non-methylated C (e.g., non-methylated CpG), or by using any other protein or protein domain that has high affinity for 5-methyl-C or 5-methyl CpG (e.g., the MBD domain of Mbd1) or non-methylated C or non-methylated CpG (e.g., the CxxC domain of MII1). The antibody or other protein with specificity for methylated/non-methylated DNA can be fused or conjugated to a reporter (such as Enhanced Green Fluorescent Protein; EGFP) or any other detectable label including fluorescence labels, radioactive labels, gold particles, and the like.

Inhibitors or activators identified in the first round of screening can optionally be evaluated further to determine the IC₅₀ and specificity using DNA demethylase assays as described herein or any other suitable assay. Compounds having a relatively low IC₅₀ and/or exhibiting specificity for a DNA of interest can be further analyzed in tissue culture and/or in a whole organism to determine their in vivo effects on DNA demethylase activity, cell proliferation, and/or toxicity.

The inventive screening methods can be cell-based or cell-free. Cell-based methods can be carried out in cultured cells and/or in whole organisms. In representative embodiments, the method provides high throughput screening assays to identify modulators of the DNA demethylase. To illustrate, a cell-based, high throughput screening assay for use in accordance with the methods disclosed herein includes that described by Stockwell et al. ((1999) Chem. Bio. 6:71-83), wherein biosynthetic processes such as DNA synthesis and post-translational processes are monitored in a miniaturized cell-based assay.

Compounds that modulate DNA demethylase activity can also be identified by identifying compounds that bind to the DNA demethylase or a component thereof (e.g., Elp1, Elp2, Elp3, Elp4, Elp5 and/or Elp6). High throughput, cell-free methods for screening small molecule libraries for candidate protein-binding molecules are well-known in the art and can be employed to identify molecules that bind to the DNA demethylase and modulate the DNA demethylase activity and/or bind to the methylated DNA substrate. For example, a methylated DNA substrate can be coated on a multi-well plate or other suitable surface and a reaction mix containing the DNA demethylase added to the substrate. Prior to, concurrent with and/or subsequent to the addition of the DNA demethylase, a test compound can be added to the well or surface containing the substrate (e.g., filter, well, matrix, bead, etc.). The reaction mixture can be washed with a solution, which optionally reflects physiological conditions to remove unbound or weakly bound test compounds. Alternatively, the test compound can be immobilized and a solution comprising the DNA demethylase can be contacted with the well, matrix, filter, bead or other surface. The ability of a test compound to modulate binding of the DNA demethylase to the substrate can be determined by any method in the art including but not limited to labeling (e.g., radiolabeling or chemiluminescence) or immunoassays (e.g., competitive ELISA assays).

Test compounds that can be screened in accordance with the methods provided herein encompass numerous chemical classes including, but not limited to, synthetic or semi-synthetic chemicals, purified natural products, proteins, antibodies, peptides, peptide aptamers, nucleic acids, oligonucleotides, carbohydrates, lipids, or other small or large organic or inorganic molecules. Small molecules are desirable because such molecules are more readily absorbed after oral administration and have fewer potential antigenic determinants. Non-peptide agents or small molecule libraries are generally prepared by a synthetic approach, but recent advances in biosynthetic methods using enzymes may enable one to prepare chemical libraries that are otherwise difficult to synthesize chemically.

Small molecule libraries can be obtained from various commercial entities, for example, SPECS and BioSPEC B.V. (Rijswijk, the Netherlands), Chembridge Corporation (San Diego, Calif.), Comgenex USA Inc., (Princeton, N.J.), Maybridge Chemical Ltd. (Cornwall, UK), and Asinex (Moscow, Russia). One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, e.g., Tan et al., (1998) Am. Chem. Soc. 120: 8565-8566; and Floyd et al., (1999) Prog Med Chem 36:91-168. Other commercially available libraries can be obtained, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. In certain embodiments of the invention, the methods are performed in a high-throughput format using techniques that are well known in the art, e.g., in multiwell plates, using robotics for sample preparation and dispensing, etc. Representative examples of various screening methods may be found, for example, in U.S. Pat. Nos. 5,985,829, 5,726,025, 5,972,621, and 6,015,692. The skilled practitioner will readily be able to modify and adapt these methods as appropriate.

A variety of other reagents can be included in the screening assays of the instant invention. These include reagents like salts, ATP, neutral proteins, e.g., albumin, detergents, etc., which can be used to facilitate optimal protein-protein and/or protein-DNA binding and/or enzymatic activity and/or reduce non-specific or background interactions. Also, reagents that otherwise improve the efficiency of the assay, such as protease inhibitors, nuclease inhibitors, anti-microbial agents, and the like may be used. The mixture of components can be added in any order that permits binding and/or enzymatic activity.

Having described the present invention, the same will be explained in greater detail in the following examples, which are included herein for illustration purposes only, and which are not intended to be limiting to the invention.

### Example 1

### Materials & Methods

### Mice and Oocyte/Zygote Preparation

All animal experiments were performed according to procedures approved by the Institutional Animal Care and Use Committee. Four to six week old BDF1 mice (C57BL6 x DBA2, Charles River) were used for all the experiments. MII oocytes, collected from female mice treated with PMSG (Harbor-UCLA) and hCG (Sigma Aldrich), were cultured in M16 medium (EmbryoMax, Millipore) at 37° C. with 5% CO₂ before being used in experiments.

### BrdU Incorporation

For in vitro fertilization (IVF), sperm and oocytes were harvested and incubated in HTF medium (EmbryoMax, Millipore) containing 30 μM BrdU (BD Pharmingen) for 3 hrs. Fertilized oocytes were treated with hyaluronidase to remove cumulus cells, and cultured in M16 medium containing BrdU until the desired PN stages. To examine the BrdU incorporation at PNO-1 stage, intracytoplasmic sperm injection (ICSI), instead of IVF, was performed. Both sperm and oocytes are incubated for 1-2 hrs in M16 medium containing 30 μM BrdU prior to ICSI.

### Immunological Detection of 5mC, BrdU, and Time-Lapse Imaging

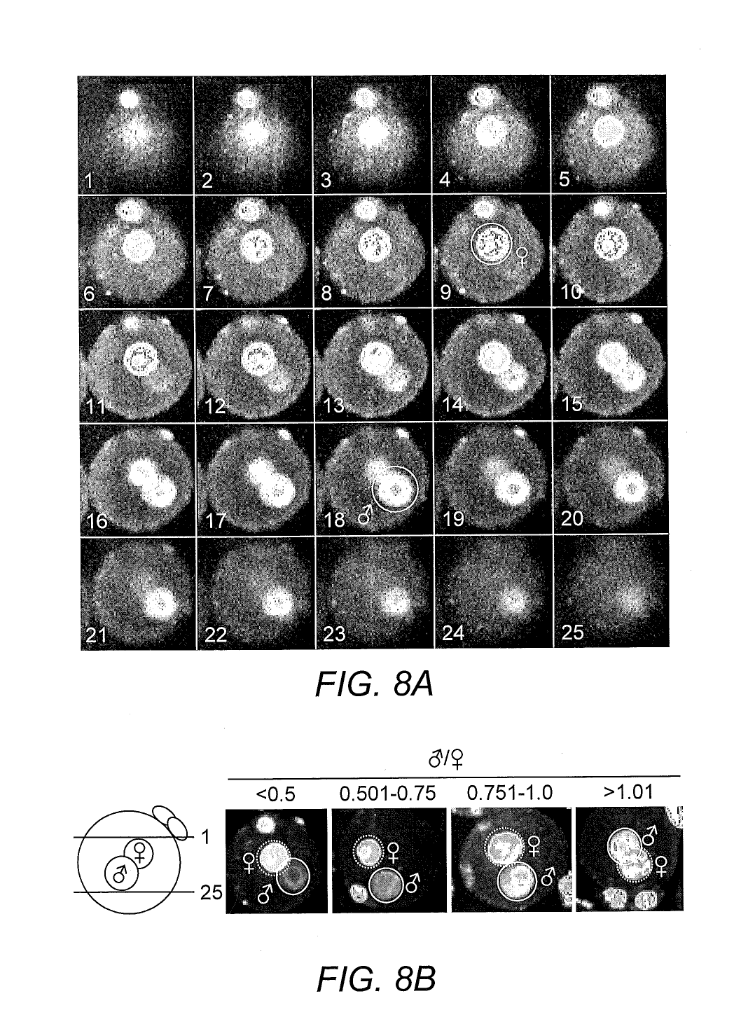

Zygotes were fixed with 4% paraformaldehyde for at least 2 hrs at 4° C. After washing with PBS, the zygotes were permeabilized with 0.4% (for 5mC) or 1% (for BrdU) TritonX-100 for 30 min at room temperature. Cells were then washed with PBS containing 0.05% Tween20 (PBST), and treated with 4N HCl for 30 min at room temperature before being neutralized with 0.1 M Tris-HCl (pH 8.5) (for 5mC) or 0.1 M sodium borate (pH 8.5) (for BrdU) for 10 min. After blocking with 1% BSA in PBST, cells were incubated with anti-5mC antibody (1:100 dilution, Eurogentic) or anti-BrdU antibody (1:100 dilution, Millipore) for 1 hr at 37° C., and the positive signal was detected by FITC-conjugated donkey anti-mouse IgG (Jackson Immmunoresearch). For DNA labeling, cells were further treated with 50 μg/ml RNase A and 5 μg/ml propidium iodine simultaneously. Fluorescent images were taken using a confocal microscope (Observer Z1, Zeiss) with a spinning disk (CSU-10, Yokogawa) and an EM-CCD camera (ImagEM, Hamamatsu). The same confocal microscope system, combined with an on-stage incubation chamber, was used for time-lapse imaging. For both live and fixed zygotes, images were acquired as multiple 2 μM-Z-axis intervals, and stacked images were reconstituted using Axiovision (Zeiss) or MetaMorph (Universal Imaging Co). The intensity of 5mC in each pronucleus was calculated by MetaMorph as shown in FIG. 8.

### DNA Constructs

cDNA that encodes the CxxC domain (aa. 1144-1250) of mouse MII1 (NCBI Accession # NP_{—}005924) was cloned by RT-PCR. cDNA for H3.3 was provided by Dr. Nakatani⁴⁶. These cDNAs were subcloned into a pcDNA3.1-poly(A)83 vector⁴⁷ with a C-terminal EGFP or mRFP1. pcDNA3.1-EGFP-MBD-poly(A)83 and pcDNA3.1-H2B-mRFP1-poly(A)83 were previously described⁴⁸. These plasmids were used for in vitro transcription using the RiboMAX Large Scale RNA production System T7 (Promega). Synthesized mRNAs were purified with Illustra MicroSpin G-25 columns (GE Healthcare) before being used for injection. The mouse Elp3 cDNA was amplified by RT-PCR and was subcloned into a pcDNA3.1-poly(A)83 vector with a Flag tag at the N-terminus. Both the Cysteine and the HAT mutants of Elp3 were generated by PCR-based mutagenesis and confirmed by sequencing. The primers used for generation of these mutants were as follows: Cys-F) 5′-ACAGGGAATATATCTATATACTCCCCCGGAGGACCTG-3′ (SEQ ID NO: 1), Cys-R) 5′-CAGGTCCTCCGGGGGAGTATATAGATATATTCCCTGT-3′ (SEQ ID NO: 2), HAT-F) 5′-AATTTCAGCATCAGTTCGCCTTCATGCTGCTGATGG-3′ (SEQ ID NO: 3), HAT-R) 5′-CCATCAGCAGCATGAAGGCGAACTGATGCTGAAATT-3′ (SEQ ID NO: 4). The underlined nucleotides are substituted in the mutants.

mRNA, siRNA, chemical Injection, RT-qPCR and Bisulfite Sequencing

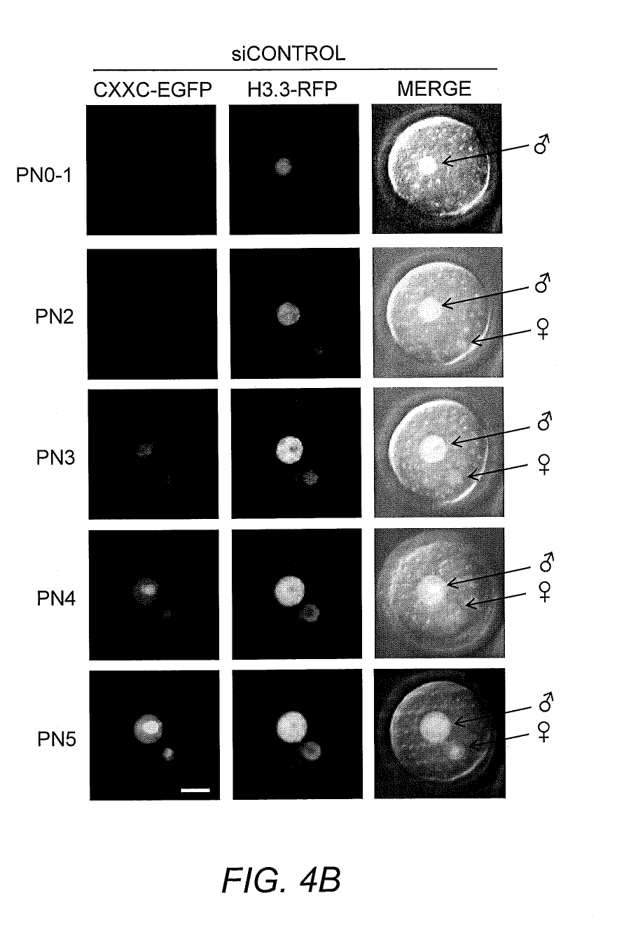

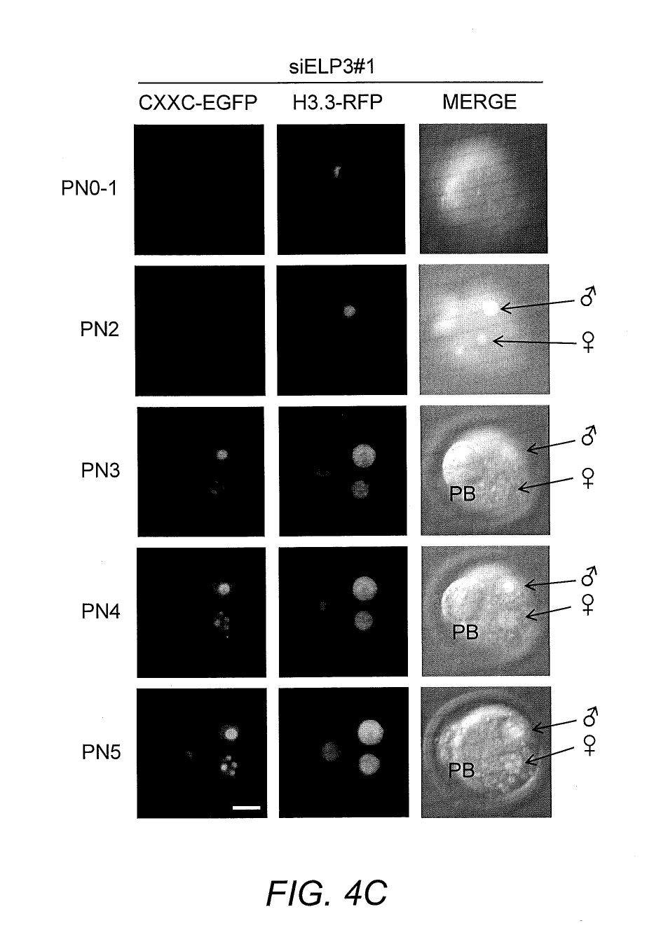

About 3-5 pl of siRNAs (2 μM) purchased from Ambion (Table 1) were co-injected with H3.3-mRFP1 (25 μg/ml) and CxxC-EGFP mRNAs (25 μg/ml) simultaneously. After 8 hrs of cultivation, cells were subjected to ICSI (FIG. 4a).

TABLE 1

| | AMBION | | SEQ |
|---|---|---|---|
| Gene | siRNA | Sense Sequence | ID |
| Name | ID# | (5′->3′) | NO: |
| Negative | n/a | No information | — |
| Control | | available (Cat# AM4611) | |
| Elp1 | s106425 | GACUGACAGGUGUCGCUUUtt | 11 |
| Elp3 #1 | s92451 | CAUCCGAAGUUUACACGAUtt | 12 |
| Elp3 #2 | s92453 | GUGUUUCCAUAGUCCGAGAtt | 13 |
| Elp4 | s211969 | GCACCACUACUUGAUGAUAtt | 14 |
| Cyp11a1 | s64660 | GCUUCGUAAUUACAAGAUUtt | 15 |
| Smc6-like | s84719 | GAUCUGCCCAGAACGGAUAtt | 16 |
| #1 | | | |
| Smc6-like | s84718 | CCGUGGUUUCUACUAGGAAtt | 17 |
| #2 | | | |
| Brm | s84569 | GAGCGAAUCCGUAAUCAUAtt | 18 |
| Alkbh5 | s113995 | ACCCUGCGCUGAAACCCAAtt | 19 |
| Nful | s80958 | GCAGUUAUUCAGAAUUGAAtt | 20 |

For chemical injection, approximately 10 pl of 3,5-Di-tert-butyltoluene and butylated hydroxytoluence (Sigma Aldrich) at the concentration of 10 μM in ethanol were injected with H3.3-mRFP1 mRNA. The final chemical concentration is estimated as

TABLE 2

| | | SEQ | | SEQ |
|---|---|---|---|---|
| | Forward | ID | Reverse | ID |
| Gene | (5′->3′) | NO: | (5′->3′) | NO: |
| 18S | CGGCTACCACATCCAA | 21 | AGCTGGAATTACCGC | 22 |
| | GGAA | | GGC | |
| Gadd45a | TGCGAGAACGACATCA | 23 | TCCCGGCAAAAACAA | 24 |
| | ACAT | | ATAAG | |
| Gadd45b | GTTCTGCTGCGACAAT | 25 | TTGGCTTTTCCAGGA | 26 |
| | GACA | | ATCTG | |
| Gadd45c | ATGACTCTGGAAGAAG | 27 | CAGGGTCCACATTCA | 28 |
| | TCCGT | | GGACT | |
| Elp1 | GAGTCAGACCTCTTCT | 29 | CGCACCTCATCTTTTA | 30 |
| | CGGAAA | | GCTTCT | |
| Elp2 | CTTTCGAAACCAAGGA | 31 | CAGAGAATCATGGTT | 32 |
| | TGGTAG | | TTGTCCA | |
| Elp3 | TCCGTGCTAGATATGA | 33 | CATCGTGTAAACTTC | 34 |
| | CCCTTT | | GGATGAA | |
| Elp4 | ACTCCCTGCACCACTA | 35 | AATCCATGCCACTTT | 36 |
| | CTTGAT | | GAACTCT | |
| Cyp11a1 | CCAGTGTCCCCATGCT | 37 | CAGCTGCATGGTCCT | 38 |
| | CAA | | TCCA | |
| Smc6- | CGTACTGAAGGGGAAT | 39 | AGGAACAGCTGGCTT | 40 |
| like | TGTGA | | TCTAGG | |
| Brm | GAGGAGGAGGAGGAA | 41 | GCTGCTTTCATCTATT | 42 |
| | GAAGAAG | | GGCTCT | |
| Alkbh5 | ACAGAGGCCTTCTAAG | 43 | CTGACCCCAAAGAGA | 44 |
| | CAGC | | CTTCC | |
| Nful | ATGGGGAGCAGCGGT | 45 | TGCGCGCAGCGGGA | 46 |
| | CGGTGTAGT | | AAAGTGGTCT | |
| H1oo | ACTGGAGATGGCACCT | 47 | TCGATTTCTCACCTTT | 48 |
| | AAGAAA | | GGTTTT | |
| MuERVL | AAATGACTTGGAGATG | 49 | TGCGTCTTATAGAGC | 50 |
| | CCTGAT | | TGGTGAA | |

0.4 μM based on an estimated mouse oocyte volume of 270 pl. The final estimated ethanol concentration is about 4%. For determination of knockdown efficiency, RNA isolated from 10-20 zygotes at PN4-5 stage was used for reverse transcription using the SuperScript III Cell Direct cDNA synthesis kit (Invitrogen) followed by quantitative PCR (qPCR) using SYBR GreenER (Invitrogen). Results were normalized with 18S rRNA as a standard. Primer sequences for qPCR are listed in Table 2.

For bisulfite sequencing, either Elp3 siRNA or control siRNA was co-injected with H3,3-mRFP1 mRNA into MII oocytes followed by ICSI after 6-8 hrs of siRNA/mRNA injection. Male pronuclei, which were distinguished from female pronuclei based on their size, distance from polar bodies, and more intense H3.3-mRFP1 fluorescence, were harvested from zygotes of PN3-4 stages by breaking the zona and cytoplasm using Piezo drive (Prime Tech), and aspirating with a micromanipulator. Forty-three male pronuclei from control siRNA-injected zygotes and 47 male pronuclei from siElp3-injected zygotes were collected, and subject to bisulfite conversion using EZ DNA Methylation-Direct Kit (Zymo Research). Nested PCR was performed using Platinum Taq DNA polymerase (Invitrogen). Both first and second-round PCRs were performed under the following conditions: 2 min at 95° C., followed by 45 cycles of PCR consisting of 30 sec at 94° C., 30 sec at 50° C., 1 min at 72° C. The sequences of the PCR primers are listed in Table 3.

TABLE 3

| | | | SEQ | | SEQ |
|---|---|---|---|---|---|
| | | Forward | ID | Reverse | ID |
| Gene | | (5′->3′) | NO: | (5′->3′) | NO: |
| Line1-5′ | 1^{st} | GTTAGAGAATTT | 51 | CCAAAACAAAACCT | 52 |
| (Ref. 27) | | GATAGTTTTTGG | | TTCTCAAACACTAT | |
| | | AATAGG | | AT | |
| | 2^{nd} | TAGGAAATTAGT | 53 | TCAAACACTATATT | 54 |
| | | TTGAATAGGTGA | | ACTTTAACAATTCC | |
| | | GAGGT | | CA | |
| ETn | 1^{st} | CTTAACTACATT | 55 | AGTTAGYGTTAGTA | 56 |
| (Ref. 26) | | TCTTCTTTTACC | | TGTGTATTTGT | |
| | 2^{nd} | TCTAAATTCCTC | 57 | | |
| | | TCTTACAACT | | | |

### Cell Culture and Transfection

Immortalized p53 knockout (KO) and p53/Dnmt1 double knockout (DKO) mouse embryonic fibroblasts (MEFs) were previously described⁴⁹. The KO MEFs, DKO MEFs, and NIH3T3 cells were maintained in DMEM supplemented with 10% FBS. pcDNA3-EGFP-pA83 plasmids containing the MBD domain and CxxC motif were transfected using Fugene6 (Roche). NIH3T3 cells that stably express CxxC-EGFP were selected under 1 mg/ml G418. 5-Aza-2′ deoxycytidine (Sigma Aldrich) was applied at the concentration of 5 μM for 72 hours.

### Example 2

### Gadd45b-Deficiency does not Affect Paternal DNA Demethylation

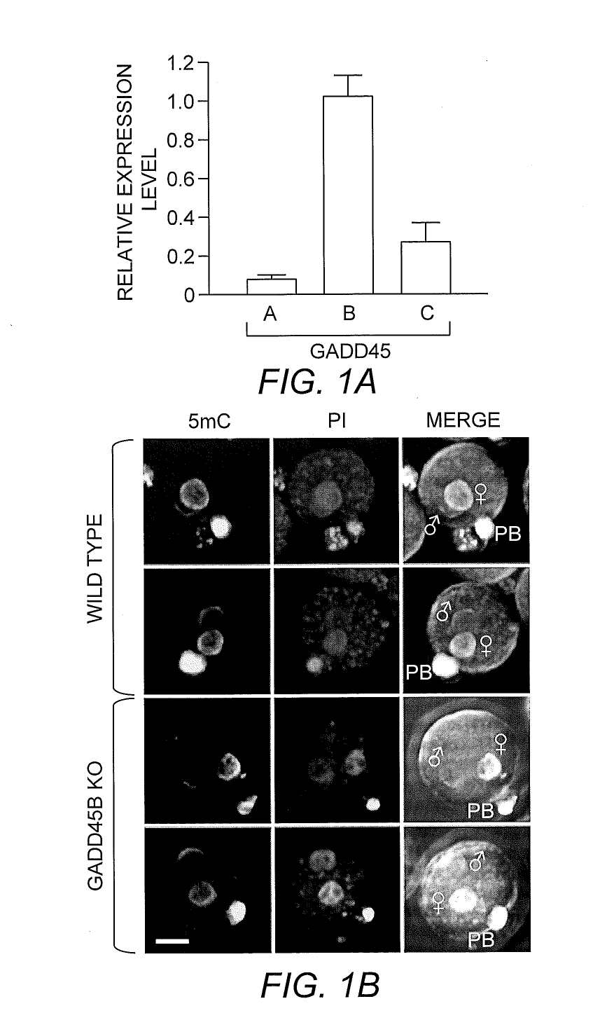

Both Gadd45a and Gadd45b have been implicated in DNA demethylation in somatic cells^{13,50}, but the role of Gadd45a in DNA demethylation has been challenged by some recent studies^{51,52}. To determine whether Gadd45 proteins play a role in paternal DNA demethylation in zygotes, we first determined the relative expression levels of the Gadd45 proteins in zygotes by real-time PCR and found that Gadd45b is the most highly expressed gene in the Gadd45 family (FIG. 1a). Because Gadd45b has been recently shown to mediate DNA demethylation in mature non-proliferating neurons⁵⁰, we asked whether loss of Gadd45b function affects zygotic paternal DNA demethylation. Results shown in FIG. 1b indicate that paternal DNA demethylation, measured by loss of 5mC Ab staining, still takes place in the Gadd45b null zygote suggesting that Gadd45b is not required for paternal DNA demethylation in zygotes.

### Example 3

### Reporter System to Monitor DNA Methylation State

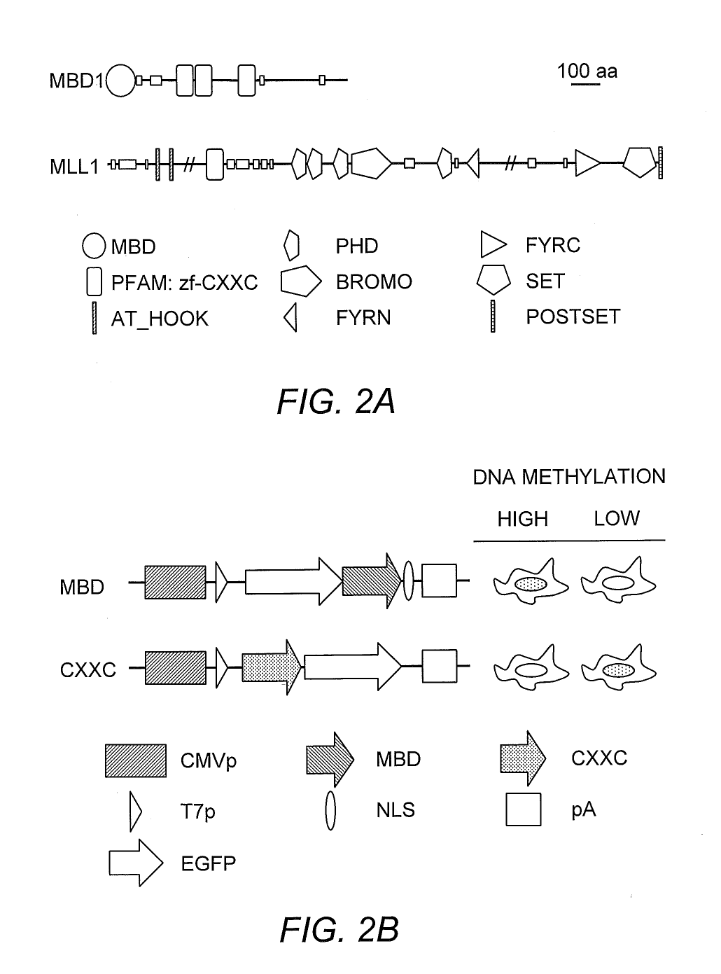

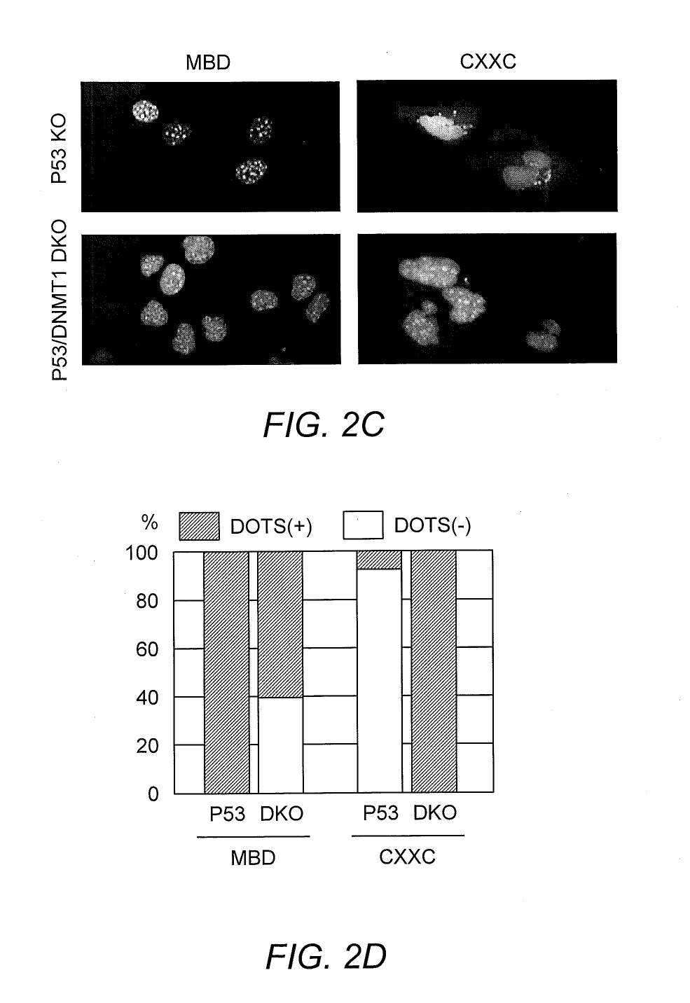

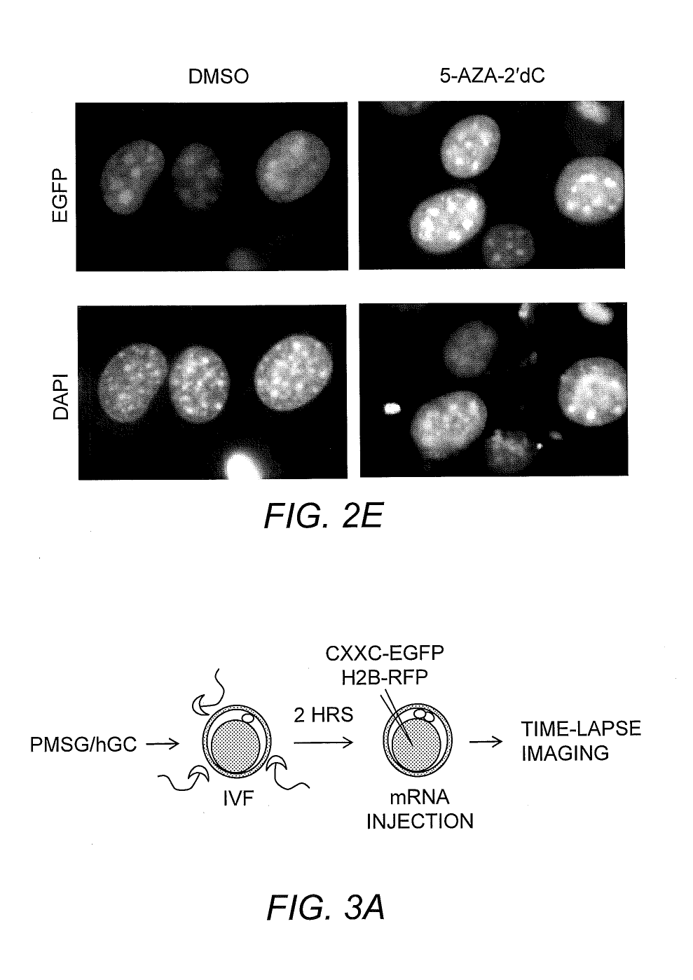

To facilitate the identification of factors involved in paternal pronuclear demethylation, we attempted to establish a system that would allow us to monitor the DNA methylation state of the zygotic paternal genome in real-time. To this end, we used a EGFP-MBD fluorescent reporter⁴⁷, as well as a new reporter constructed by fusing the CxxC domain of the MII1 protein to EGFP (FIG. 2a, b). The MBD domain of Mbd1 and the CxxC domain of MII1 have high affinity to methyl-CpG and non-methyl-CpG, respectively^{53,54}, and therefore we expect that the subnuclear distribution of these reporters might serve as an indicator of DNA methylation state in living cells. To evaluate the potential of these fusion proteins to serve as an indicator of DNA methylation state, plasmids that encode the fusion proteins were transfected into mouse fibroblasts with normal CpG methylation (p53 KO) or without CpG methylation (p53/Dnmt1 DKO). As expected, EGFP-MBD exhibited a nuclear dotted pattern, while CxxC-EGFP exhibited diffused nuclear staining in cells with normal CpG methylation (FIG. 2c, d). In contrast, almost 100% of cells without CpG methylation exhibited punctate nuclear localization of CxxC-EGFP. Unexpectedly, the nuclear dotted pattern of EGFP-MBD was still maintained in ˜60% of the DKO cells (FIG. 2c, d). Intense DAPI staining indicates that the nuclear dots correlate to mouse satellite DNA which is enriched for 5mCpG. This result indicates that when compared to EGFP-MBD, CxxC-EGFP is a better reporter whose changes in distribution can better indicate a change in DNA methylation state. We further confirmed the utility of the CxxC-EGFP reporter in NIH3T3 cells by demonstrating that 5-Aza-dC-mediated DNA demethylation resulted in a clear increase in the number, as well as intensity, of GFP bright dots (FIG. 2e). These results suggest that CxxC-EGFP can serve as an indicator of DNA methylation state in living cells.

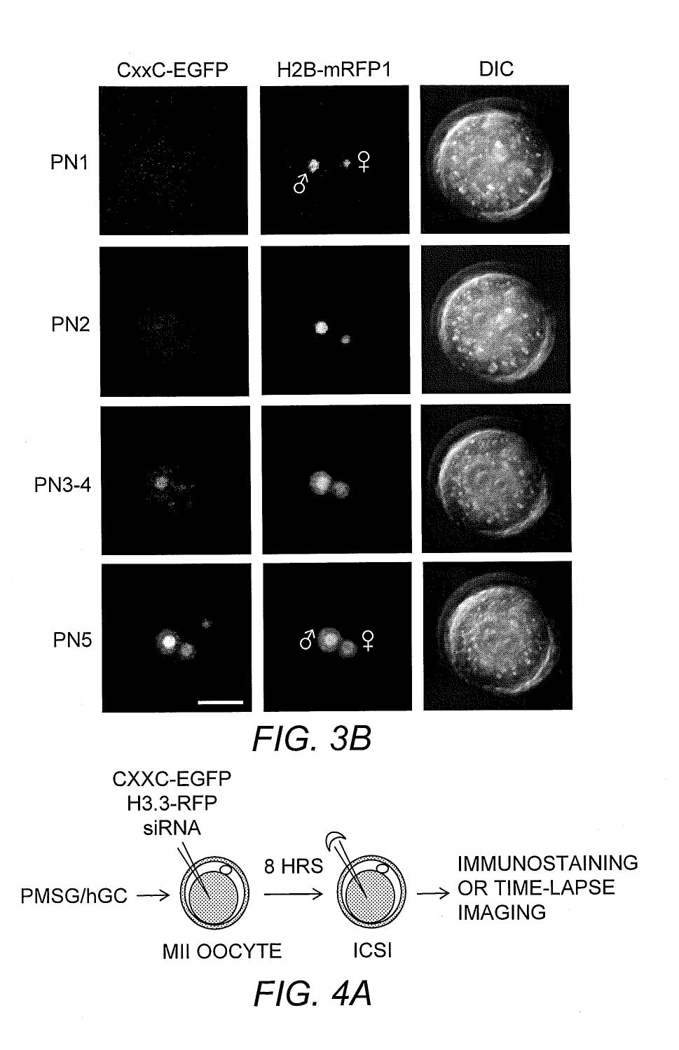

We next examined whether the CxxC-EGFP reporter can accurately “report” paternal genome demethylation by enriching asymmetrically in the paternal PN. Since at least 10 hours were required for injected plasmid DNA to be expressed in zygotes, injection of the CxxC-EGFP plasmid DNA would not allow the paternal PN demethylation process to be monitored. Therefore, we adapted a previously published mRNA injection technique that allows visualization of molecular events in the mammalian zygote as early as 3 hours after introduction⁴⁷. Poly(A) mRNA for the CxxC-EGFP was generated using in vitro transcription with T7 polymerase (FIG. 2b). We also generated mRNA for H₂B-mRFP1 (monomeric red fluorescent protein 1) to serve as a marker for PNs. Using the procedure outlined in FIG. 3a, we co-injected mRNAs that encode H₂B-mRFP1 and CxxC-EGFP into the zygotes immediately after in vitro fertilization (IVF). Time-lapse imaging of the injected zygotes indicated that CxxC-EGFP is visible at PN2 stage and accumulates throughout the PN3-4 and PN5 stages (FIG. 3b). When compared with paternal PN, the maternal PN exhibits very little CxxC-EGFP accumulation (FIG. 3b). The dynamics of paternal PN CxxC-EGFP accumulation mimics paternal DNA demethylation dynamics reported previously^{1,2}. Based on this result, we conclude that paternal genome demethylation can be monitored by injection of CxxC-EGFP mRNA in zygotes.

### Example 4

### Elp3 is Involved for Paternal DNA Demethylation

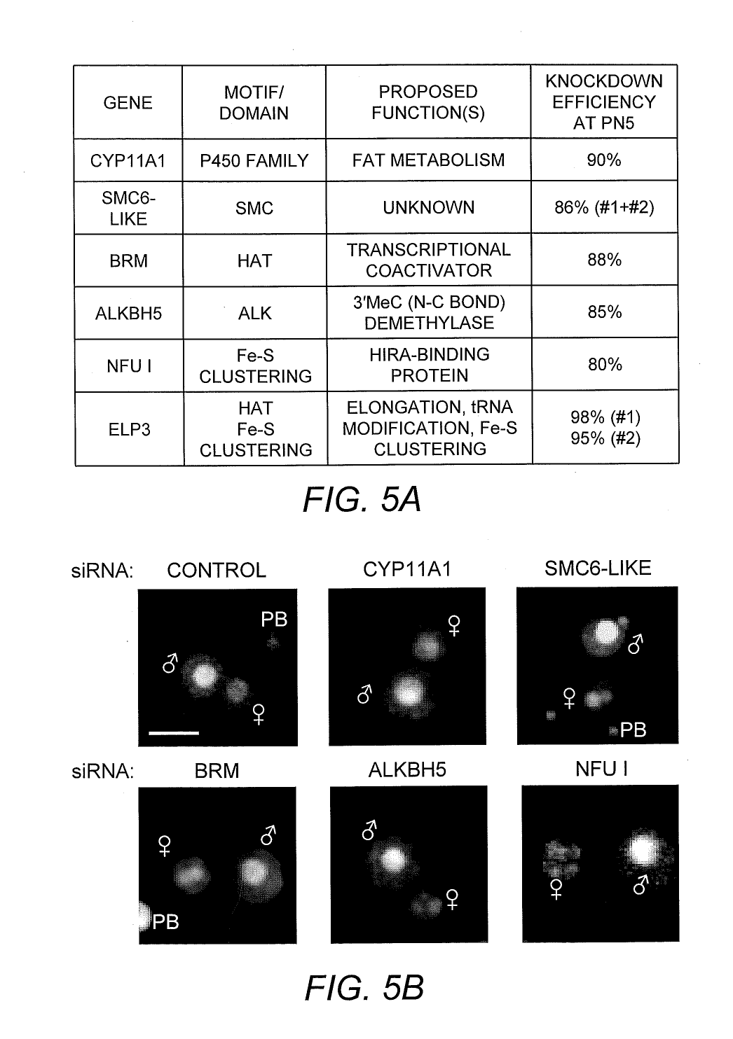

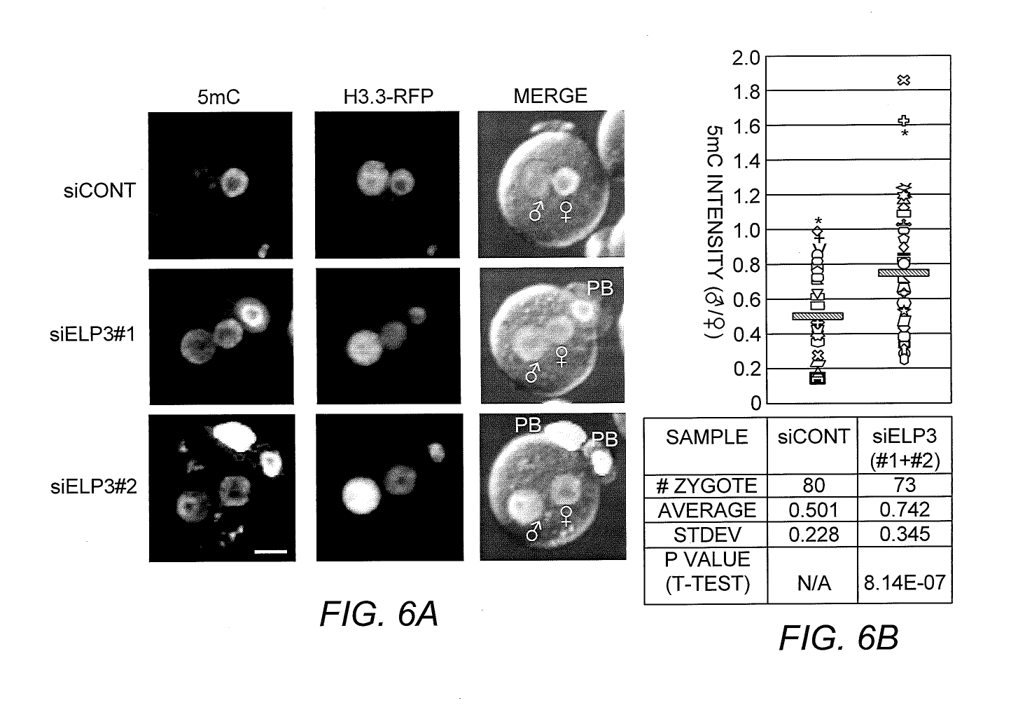

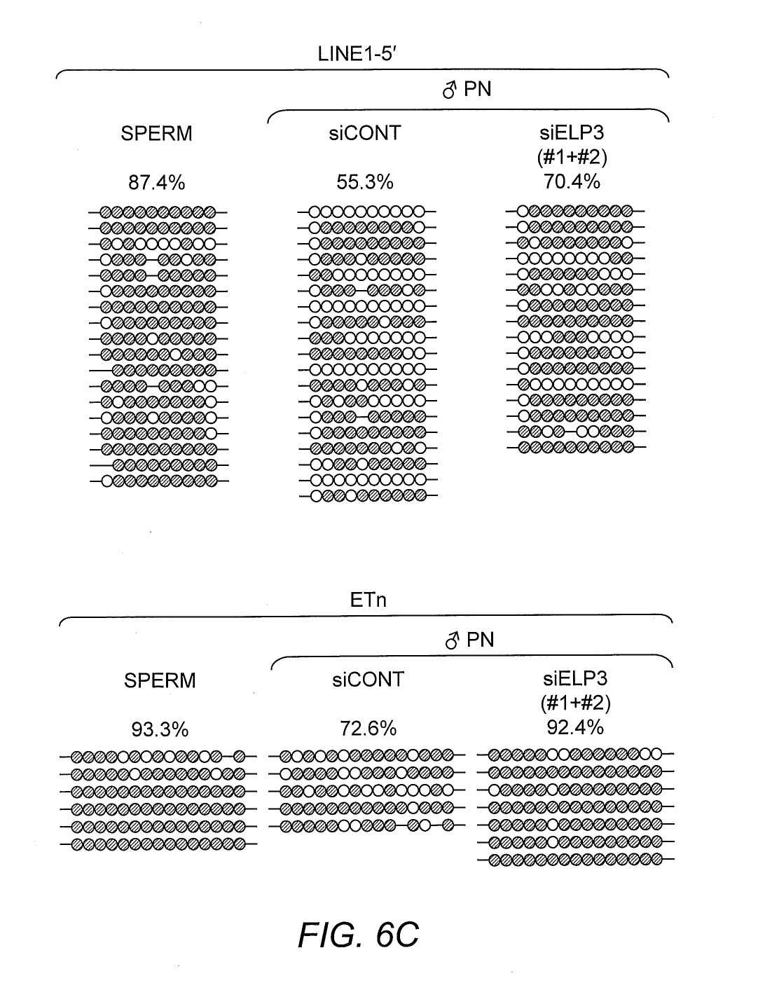

Having a reporter system established, we next asked whether siRNA-mediated depletion of candidate mRNAs in the oocytes could affect DNA demethylation during zygotic development. To this end, we first determined the optimal siRNA concentration and the time needed for injected siRNA to become effective using siRNA against Lamin A/C. Based on a previous report⁵⁵, we tested a range of siRNA concentrations (0.1-10 μM) as well as several PN staged time points (data not shown). Based on trial results, we found that the minimum dose and incubation time prior to intra-cellular sperm injection (ICSI) for effective knockdown is 2 μM and 8 hr, respectively. Given that there are about 13 hrs from the time of siRNA injection to the time of paternal DNA demethylation at PN3, the modified experimental procedure, outlined in FIG. 4a, will allow siRNA to be fully effective. In addition, we also facilitated early stage PN identification (PNO-2) by taking advantage of the preferential deposition of H3.3 into the paternal PN following fertilization⁵⁶ through the use of H3.3-mRFP1. This modified experimental scheme allowed us to monitor H3.3 deposition and DNA demethylation simultaneously with time-lapse imaging. FIG. 4b is a representative snap shot of the various PN stages with the injection of a scrambled siRNA control. This time-lapse imaging system coupled with siRNA knockdown, allowed us to test a dozen candidate genes selected based on several criteria that include: 1) their expression in zygotes; 2) the domain/structure motifs they contain; and 3) their potential in catalyzing the DNA demethylation reaction. Using these criteria, we designed siRNAs that target candidate genes including the recently identified 5mC hydroxylase Teti (Tahiliani et al., (2009) Science 324:930-935). However, we achieved more than 80% of knockdown efficiency in only six of the candidate genes (FIG. 5a). While knockdown on the majority of the candidate genes does not alter the heavily paternal pronucleus preferential distribution of the reporter (FIG. 5b), the asymmetric distribution pattern is greatly diminished upon knockdown of Elp3 (FIG. 4c). To verify this preliminary observation, we used immunostaining with the anti-5mC antibody. Results shown in FIG. 6a clearly demonstrate that knockdown of Elp3 prevents paternal DNA from demethylation. Furthermore, a second siRNA that targets a different region of Elp3 also resulted in a similar result. These results collectively indicate that Elp3 is important for paternal DNA demethylation in zygotes.

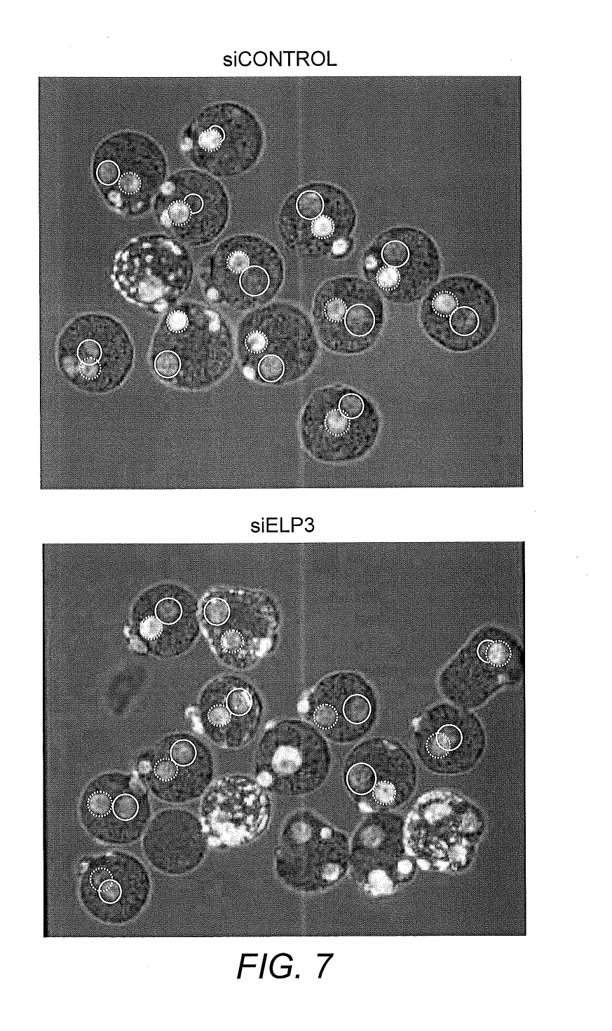

Although preferential demethylation of the paternal genome in zygotes is a general phenomenon, the extent of demethylation of individual zygotes is variable (FIG. 7). Therefore, we decided to quantitatively evaluate the effect of Elp3 knockdown on paternal DNA demethylation by analyzing a large number of zygotes (FIG. 7). To this end, one Z-section which contains the highest 5mC staining intensity of either male or female PN was selected among serial Z-axis images (2 mm interval) for quantification (FIG. 8a). A ratio of paternal over maternal 5mC intensity was determined for each zygote (FIG. 8b). Analysis of 80 PN4-5 stage zygotes with control injection results in an average ratio of 0.501. However, this ratio is significantly increased (p value of 8.14E-07) with injection of siRNAs that target Elp3 (FIG. 6b). These results indicate that Elp3 knockdown significantly impairs paternal DNA demethylation as judged by 5mC Ab staining.

To provide direct evidence that Elp3 knockdown affects paternal DNA demethylation, we evaluated DNA methylation levels by bisulfite sequencing. Previous studies have demonstrated that the transposable elements Line-1 and Etn (early retrotransposons) are subject to demethylation in zygotes^{57,58}. We therefore asked whether knockdown of Elp3 would impair their demethylation. To this end, we injected siRNAs that target Elp3 prior to ICSI and isolated paternal pronuclei at the PN3-4 stages when the DNA demethylation is at the beginning or is still occurring. We note that this is the latest time that we can still isolate paternal pronuclei without co-isolating the maternal pronuclei as the two pronuclei come too close at PN5 stage. Despite the fact that demethylation is far from completion at the PN³-4 stage, knockdown of Elp3 still clearly affected both Line-1 and Etn demethylation (FIG. 6c). Based on data from CxxC-EGFP reporter assay, 5mC Ab staining, and bisulfite sequencing, we conclude that Elp3 plays an important role in paternal DNA demethylation.

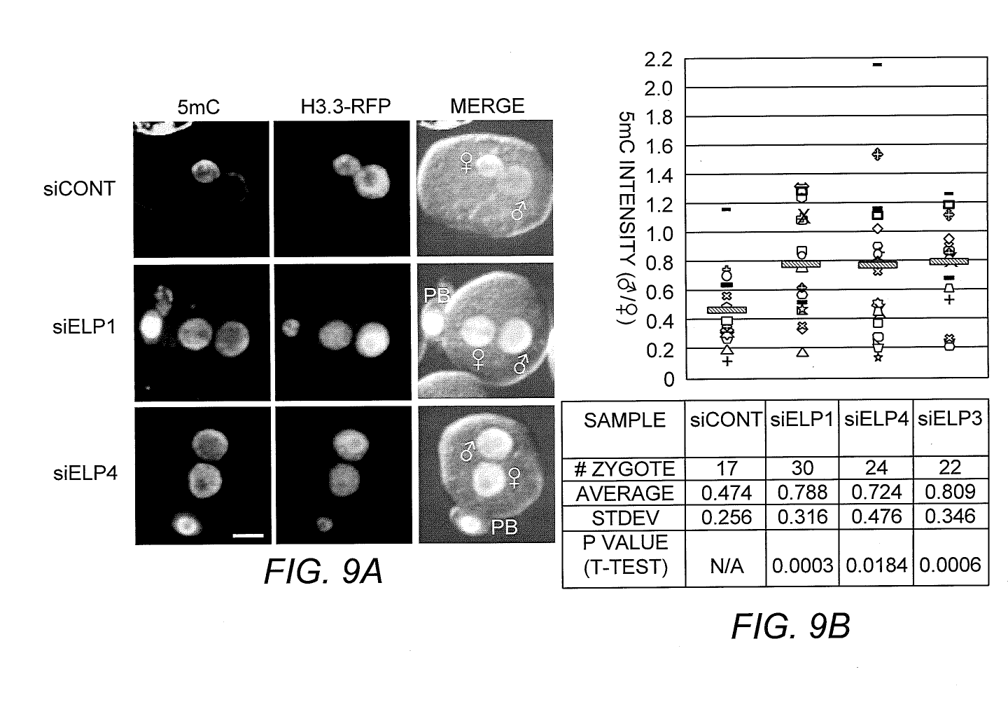

Elp3 is a component of the elongator complex that was initially identified based on its association with the RNA polymerase II holoenzyme involved in transcriptional elongation⁵⁹. Subsequent studies have revealed that the elongator complex has diverse functions that include cytoplasmic kinase signalling, exocytosis, and tRNA modification¹⁷. The yeast elongator complex is composed of six subunits, Elp1-6, that include the histone acetyltransferase (HAT) Elp3⁶⁰. The human elongator purified from HeLa is also composed of six subunits²⁰. To determine whether knockdown of other elongator subunits in oocytes also prevents paternal DNA from demethylation, we performed knockdown on two additional elongator subunits, Elp1 and Elp4. Results shown in FIG. 9 demonstrate knockdown of these two proteins also prevented paternal genome demethylation.

### Example 5

### Paternal DNA Demethylation is Mediated Through the Radical SAM Motif of Elp3

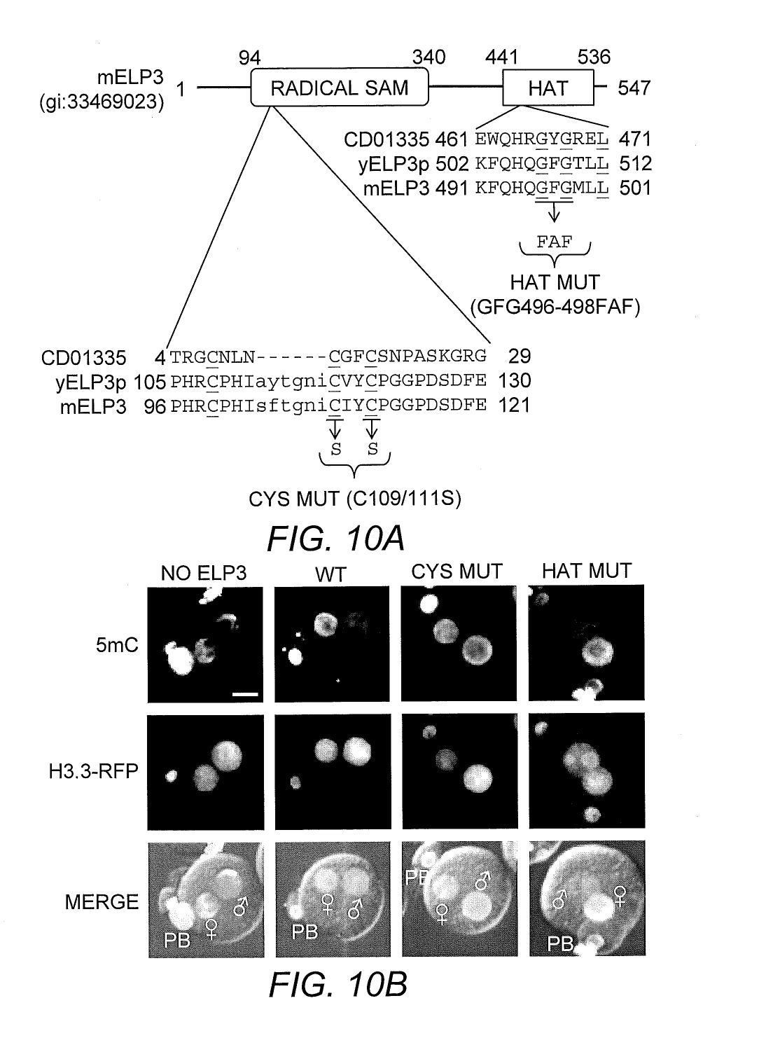

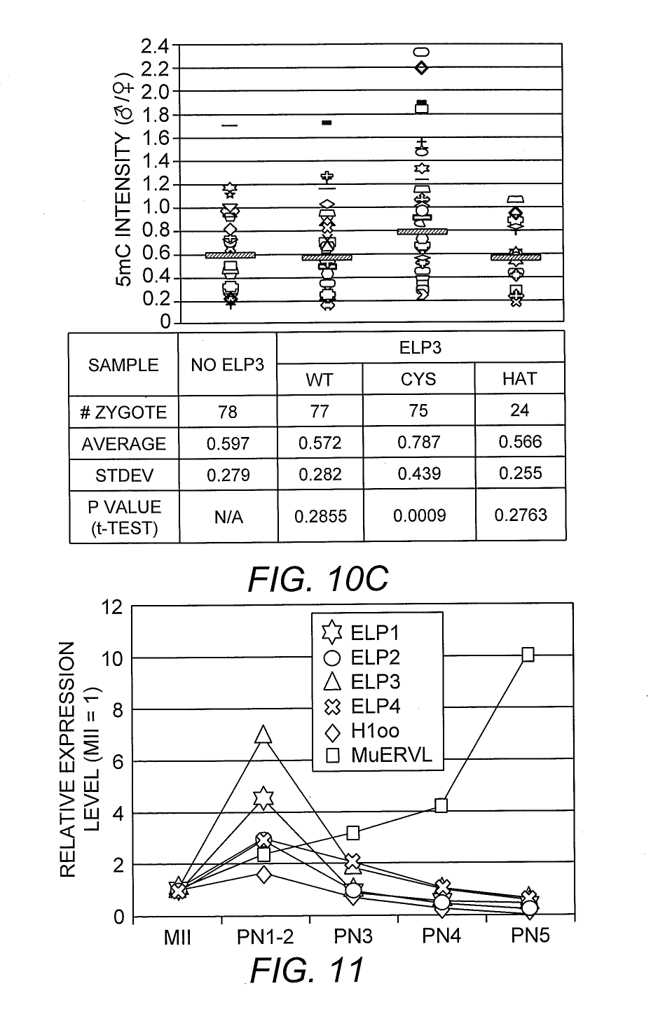

In addition to a conserved HAT domain, Elp3 also contains another conserved domain that shares significant sequence homology with the Radical SAM superfamily (FIG. 10a). Members of this superfamily contain an iron-sulfur (Fe—S) cluster and use S-adenosylmethionine (SAM) to catalyze a variety of radical reactions⁶¹. Interestingly, a recent study confirmed the presence of this Fe₄S₄ cluster in the bacteria Methanocaldococcus jannaschii Elp3 protein⁵⁹. To determine whether any of the two conserved domains of the mouse Elp3 are important for paternal DNA demethylation, we used a dominant negative approach and generated mRNAs that harbor mutations in the cysteine-rich motif (part of the F—S Radical SAM motif) and the HAT domain, respectively (FIG. 10a). As a control, we also generated wild-type Elp3 mRNA. Injection of the cysteine mutant mRNA, but not the wild-type or HAT mutant mRNA, significantly impaired paternal DNA demethylation (FIG. 10b,c), indicating that the cysteine-rich motif, but not the HAT domain, is involved in paternal genome demethylation.

### Example 6

### Summary

Whether DNA methylation is an enzymatically reversible reaction in vertebrates has been the subject of extensive study and also some controversy⁷. Although several recent reports have implicated the involvement of DNA repair proteins in DNA demethylation reactions^{12,13,14} none of them have been shown to be required for the paternal genome demethylation in zygotes.

Using a live cell imaging reporter system coupled with siRNA knockdown, we uncovered a central function of the elongator complex in mediating paternal DNA demethylation. Several lines of evidence support our conclusion. First, three independent assays (reporter, 5mC staining, bisulfite sequencing) indicate that knockdown of Elp3 impairs paternal DNA demethylation (FIGS. 4, 6). Second, knockdown of additional components of the elongator complex Elp1 and Elp4 also impaired paternal DNA demethylation (FIG. 9). Third, a dominant negative approach identified the radical SAM domain, but not the HAT domain, of Elp3 to be critical for the demethylation to occur (FIG. 10). Consistent with the involvement of the elongator complex in zygote demethylation, the RNA levels of Elp1-4 are up-regulated 3-9 fold in the PN1-2 stages prior to the start of paternal DNA demethylation at PN3 (FIG. 11).

The fact that the radical SAM domain is required for demethylation to occur points to a potential mechanism that involves the generation of a powerful oxidizing agent, 5′-deoxyadenosyl radical, from SAM. 5′-deoxyadenosyl radical can then extract a hydrogen atom from the methyl group of 5mC to generate 5mC radical for subsequent reactions. Confirmation of this proposed mechanism will be facilitated by the demonstration of enzymatic activity in vitro using recombinant proteins.

### Example 7

### Elp1 and Elp3 Knockout Mice

Knock-out mice have been generated for Elp1 and Elp3. These animals are used to confirm the observations in knockdown experiments using Elp1 or Elp3 deficient eggs and/or to analyze the effect of defective paternal DNA demethylation on development.

The foregoing is illustrative of the present invention, and is not to be construed as limiting thereof. The invention is defined by the following claims, with equivalents of the claims to be included therein.

### REFERENCES

1. Mayer, W., Niveleau, A., Walter, J., Fundele, R. & Haaf, T. Demethylation of the zygotic paternal genome. Nature 403, 501-502 (2000).
2. Oswald, J. et al. Active demethylation of the paternal genome in the mouse zygote. Curr Biol 10, 475-478 (2000).
3. Howell, C. Y. et al. Genomic imprinting disrupted by a maternal effect mutation in the Dnmt1 gene. Cell 104, 829-838 (2001).
4. Hajkova, P. et al. Epigenetic reprogramming in mouse primordial germ cells. Mech Dev 117, 15-23 (2002).
5. Sasaki, H. & Matsui, Y. Epigenetic events in mammalian germ-cell development: reprogramming and beyond. Nat Rev Genet. 2008, 129-140 (2008).
6. Simonsson, S. & Gurdon, J. DNA demethylation is necessary for the epigenetic reprogramming of somatic cell nuclei. Nat Cell Biol 6, 984-990 (2004).
7. Ooi, S. K. & Bestor, T. H. The colorful history of active DNA demethylation. Cell 133, 1145-1148 (2008).
8. Bhattacharya, S. K., Ramchandani, S., Cervoni, N. & Szyf, M. A mammalian protein with specific demethylase activity for mCpG DNA. Nature 397, 579-583 (1999).
9. Santos, F., Hendrich, B., Reik, W. & Dean, W. Dynamic reprogramming of DNA methylation in the early mouse embryo. Dev Biol 241, 172-182 (2002).
10. Choi, Y. et al. DEMETER, a DNA glycosylase domain protein, is required for endosperm gene imprinting and seed viability in arabidopsis. Cell 110, 33-42 (2002).
11. Gong, Z. et al. ROS1, a repressor of transcriptional gene silencing in Arabidopsis, encodes a DNA glycosylase/lyase. Cell 111, 803-814 (2002).
12. Rai, K. et al. DNA demethylation in zebrafish involves the coupling of a deaminase, a glycosylase, and gadd45. Cell 135, 1201-1212 (2008).
13. Barreto, G. et al. Gadd45a promotes epigenetic gene activation by repair-mediated DNA demethylation. Nature 445, 671-675 (2007).
14. Metivier, R. et al. Cyclical DNA methylation of a transcriptionally active promoter. Nature 452, 45-50 (2008).
15. Gehring, M., Reik, W. & Henikoff, S. DNA demethylation by DNA repair. Trends Genet. 25, 82-90 (2009).
16. Otero, G. et al. Elongator, a multisubunit component of a novel RNA polymerase II holoenzyme for transcriptional elongation. Mol Cell 3, 109-118 (1999).
17. Svejstrup, J. Q. Elongator complex: how many roles does it play? Curr Opin Cell Biol 19, 331-336 (2007).
18. Wittschieben, B. O. et al. A novel histone acetyltransferase is an integral subunit of elongating RNA polymerase II holoenzyme. Mol Cell 4, 123-128 (1999).
19. Hawkes, N. A. et al. Purification and characterization of the human elongator complex. J Biol Chem 277, 3047-3052 (2002).
20. Chinenov, Y. A second catalytic domain in the Elp3 histone acetyltransferases: a candidate for histone demethylase activity? Trends Biochem Sci 27, 115-117 (2002).
21. Greenwood, C. et al. An iron-sulfur cluster domain in Elp3 important for the structural integrity of Elongator. J Biol Chem 284, 141-149 (2009).
22. Reik, W. Stability and flexibility of epigenetic gene regulation in mammalian development. Nature 447, 425-432 (2007).
23. Oswald, J. et al. Active demethylation of the paternal genome in the mouse zygote. Current Biology 10, 475-478 (2000).
24. Ooi S. K. and T. H. Bestor. The colorful history of active DNA demethylation. Cell 133, 1145-1148 (2008).
25. Hawkes, N. A. et al. Purification and characterization of the human elongator complex. J Biol Chem 277, 3047-3052 (2002).
26. Altschul, S. F. et al. Gapped BLAST and PSI-BLAST: a new generation of protein database search programs. Nucleic Acids Res 25(17), 3389-3402 (1997).
27. Wu G. Y. and C. H. Wu. Receptor-mediated gene delivery and expression in vivo. J Biol Chem 263, 14621-14624 (1988).
28. Wilson, J. M. et al. Hepatocyte directed gene transfer in vivo leads to transient improvement of hypercholesterolemia in low density lipoprotein receptor-deficient rabbits. J Biol Chem 267, 963-967 (1992).
29. Feigner, P. L. et al. Lipofection: a highly efficient lipid-mediated DNA transfection procedure. Proc Natl Acad Sci USA 84, 7413-7417 (1987).
30. Machy, P. et al. Gene transfer from targeted liposomes to specific lymphoid cells by electroporation. Proc Natl Acad Sci USA 85, 8027-8031 (1988)
31. Ulmer, J. B. et al. Heterologous protection against influenza by injection of DNA encoding a viral protein. Science 259, 1745-1749 (1993).
32. Feigner, P. L. and G. M. Ringold. Cationic liposome-mediated transfection. Nature 337, 387-388 (1989).
33. Curiel, D. T. et al. High efficiency in vitro gene transfer mediated by adenovirus coupled to DNA-polylysine complexes. Hum Gene Ther 3, 147-154 (1992).
34. Wu G. Y. and C. H. Wu. Receptor-mediated in vitro gene transformation by a soluble DNA carrier system. J Biol Chem 262, 4429-4432 (1987).
35. Kim, J.-H. et al. Human Elongator facilitates RNA polymerase II transcription through chromatin. Proc Natl Acad Sci USA 99, 1241-1246 (2002).
36. Kim, S.-H. and T. R. Cech. Three-dimensional model of the active site of the self-splicing rRNA precursor of Tetrahymena. Proc Natl Acad Sci USA 84, 8788-8792 (1987).
37. Gerlach, W. L. et al. Construction of a plant disease resistance gene from the satellite RNA of tobacco ringspot virus. Nature 328, 802-805 (1987).
38. Forster, A. C. and R. H. Symons. Self-cleavage of plus and minus RNAs of a virusoid and a structural model for the active sites. Cell 49, 211-220 (1987).
39. Michel F. and E. Westhof. Modelling of the three-dimensional architecture of group I catalytic introns based on comparative sequence analysis. J Biol Chem 216, 585-610 (1990).
40. Reinhold-Hurek, B. and D. A. Shub. Self-splicing introns in tRNA genes of widely divergent bacteria. Nature 357, 173-176 (1992).
41. Joyce, G. F. RNA evolution and the origins of life. Nature 338. 217-224 (1989).
42. Scanlon, K. J. et al. Ribozyme-mediated cleavage of c-fos mRNA reduces gene expression of DNA synthesis enzymes and metallothionein. Proc Natl Acad Sci USA 88, 10591-10595 (1991).
43. Sarver, N. et al. Ribozymes as potential anti-HIV-1 therapeutic agents. Science 247, 1222-1225 (1990).
44. Sioud, M. et al. Preformed Ribozyme Destroys Tumor Necrosis Factor mRNA in Human Cells. J Mol Biol 223, 831-835 (1992).
45. Couzin, J. MicroRNAs Make Big Impression in Disease After Disease. Science 319, 1782-1784 (2008).
46. Tagami, H., Ray-Gallet, D., Almouzni, G. & Nakatani, Y. Histone H3.1 and H3.3 complexes mediate nucleosome assembly pathways dependent or independent of DNA synthesis. Cell 116, 51-61 (2004).
47. Yamagata, K. et al. Noninvasive visualization of molecular events in the mammalian zygote. Genesis 43, 71-79 (2005).
48. Yamazaki, T., Yamagata, K. & Baba, T. Time-lapse and retrospective analysis of DNA methylation in mouse preimplantation embryos by live cell imaging. Dev Biol 304, 409-419 (2007).
49. Jackson-Grusby, L. et al. Loss of genomic methylation causes p53-dependent apoptosis and epigenetic deregulation. Nat Genet. 27, 31-39 (2001).
50. Ma, D. K. et al. Neuronal activity-induced Gadd45b promotes epigenetic DNA demethylation and adult neurogenesis. Science 323, 1074-1077 (2009).
51. Engel, N. et al. Conserved DNA demethylation in Gadd45a(−/−) mice. Epigenetics 4, 98-99 (2009).
52. Jin, S. G., Guo, C. & Pfeifer, G. P. Gadd45A does not promote DNA demethylation. PLoS Genet. 4, e1000013 (2008).
53. Allen, M. D. et al. Solution structure of the nonmethyl-CpG-binding CXXC domain of the leukaemia-associated MLL histone methyltransferase. Embo J 25, 4503-4512 (2006).
54. Jorgensen, H. F., Adie, K., Chaubert, P. & Bird, A. P. Engineering a high-affinity methyl-CpG-binding protein. Nucleic Acids Res 34, e96 (2006).
55. Amanai, M., Shoji, S., Yoshida, N., Brahmajosyula, M. & Perry, A. C. Injection of mammalian metaphase II oocytes with short interfering RNAs to dissect meiotic and early mitotic events. Biol Reprod 75, 891-898 (2006).
56. Torres-Padilla, M. E., Bannister, A. J., Hurd, P. J., Kouzarides, T. & Zernicka-Goetz, M. Dynamic distribution of the replacement histone variant H3.3 in the mouse oocyte and preimplantation embryos. Int J Dev Biol 50, 455-461 (2006).
57. Kim, S. H. et al. Differential DNA methylation reprogramming of various repetitive sequences in mouse preimplantation embryos. Biochem Biophys Res Commun 324, 58-63 (2004).
58. Lane N. et al. Resistance of IAPs to methylation reprogramming may provie a mechanism for epigenetic inheritance in the mouse. Genesis 35, 88-93 (2003).
59. Paraskevopoulou, C., Fairhurst, S. A., Lowe, D. J., Brick, P. & Onesti, S. The Elongator subunit Elp3 contains a Fe4S4 cluster and binds S-adenosylmethionine. Mol Microbiol 59, 795-806 (2006).
60. Tong, W. H., Jameson, G. N., Huynh, B. H. & Rouault, T. A. Subcellular compartmentalization of human Nfu, an iron-sulfur cluster scaffold protein, and its ability to assemble a [4Fe-4S] cluster. Proc Natl Acad Sci USA 100, 9762-9767 (2003).
61. Wang, S. C. & Frey, P. A. S-adenosylmethionine as an oxidant: the radical SAM superfamily. Trends Biochem Sci 32, 101-110 (2007).

1. A recombinant mammalian DNA demethylase comprising Elp3.

2. The recombinant DNA demethylase of claim 1, wherein the DNA demethylase comprises a complex comprising Elp1, Elp3 and Elp4.

3. An isolated mammalian DNA demethylase comprising Elp3.

4. The isolated DNA demethylase of claim 3, wherein the DNA demethylase comprises a complex comprising Elp1, Elp3 and Elp4.

5. The DNA demethylase of claim 1, wherein the DNA demethylase comprises a complex further comprising one or more of Elp2, Elp5 or Elp6.

6. The DNA demethylase of claim 1, wherein the DNA demethylase is a human DNA demethylase.

7. A method of reducing DNA methylation in a mammalian cell, the method comprising introducing the DNA demethylase according to claim 1 into the cell.

8. The method of claim 7, wherein the Elp3 is a recombinant Elp3.

9. The method of claim 7, wherein the Elp3 is a mammalian Elp3.

10. The method of claim 9, wherein the Elp3 is a human Elp3.

11. The method of claim 8, wherein the Elp3 is a recombinant Elp3 and nucleic acid encoding Elp3 is injected into the cell.

12-24. (canceled)

25. A method of reducing DNA demethylation in a mammalian cell, the method comprising reducing the activity of Elp1, Elp2, Elp3, Elp4, Elp5 or Elp6, or any combination thereof, in the cell.

26-40. (canceled)

41. A method of preventing or treating cancer in a mammalian subject in need thereof, the method comprising reducing the activity of Elp1, Elp2, Elp3, Elp4, Elp5 or Elp6, or any combination thereof, in the subject.

42-44. (canceled)

45. A method of modifying a transcriptional program in a mammalian cell, the method comprising introducing Elp3 into the cell.

46-57. (canceled)

58. A method of identifying a compound that modulates the DNA demethylase activity of recombinant mammalian Elp3, the method comprising:
(a) contacting a recombinant mammalian Elp3 with a DNA substrate in the presence of a test compound; and
(b) detecting the level of demethylation of the DNA substrate under conditions sufficient for DNA demethylation, wherein a change in demethylation of the DNA substrate as compared with the level of demethylation in the absence of the test compound indicates that the test compound is a modulator of the DNA demethylase activity of Elp3.

59. A method of identifying a compound that modulates the DNA demethylase activity of a recombinant mammalian complex comprising Elp1, Elp3 and Elp4, the method comprising:
(a) contacting the recombinant mammalian complex with a DNA substrate in the presence of a test compound; and
(b) detecting the level of demethylation of the DNA substrate under conditions sufficient for DNA demethylation, wherein a change in demethylation of the DNA substrate as compared with the level of demethylation in the absence of the test compound indicates that the test compound is a modulator of the DNA demethylase activity of the complex.

60-62. (canceled)

63. A method of identifying a candidate compound for the treatment of cancer, the method comprising:
(a) contacting a recombinant mammalian Elp3 with a DNA substrate in the presence of a test compound; and
(b) detecting the level of demethylation of the DNA substrate under conditions sufficient for DNA demethylation, wherein a change in demethylation of the DNA substrate as compared with the level of demethylation in the absence of the test compound indicates that the test compound is a candidate compound for the treatment of cancer.

64. A method of identifying a candidate compound for the treatment of cancer, the method comprising:
(a) contacting a recombinant mammalian complex comprising Elp1, Elp3 and Elp4 with a DNA substrate in the presence of a test compound; and
(b) detecting the level of demethylation of the DNA substrate under conditions sufficient for DNA demethylation, wherein a change in demethylation of the DNA substrate as compared with the level of demethylation in the absence of the test compound indicates that the test compound is a candidate compound for the treatment of cancer.

65-67. (canceled)

68. A method of identifying a candidate compound for the modulation of gene expression in a cell, the method comprising:
(a) contacting a recombinant mammalian Elp3 with a DNA substrate in the presence of a test compound; and
(b) detecting the level of demethylation of the DNA substrate under conditions sufficient for DNA demethylation, wherein an increase in demethylation of the DNA substrate as compared with the level of demethylation in the absence of the test compound indicates that the test compound is a candidate compound for modulating gene expression in a cell.

69. A method of identifying a candidate compound for modulating gene expression in a cell, the method comprising:
(a) contacting a recombinant mammalian complex comprising Elp1, Elp3 and Elp4 with a DNA substrate in the presence of a test compound; and
(b) detecting the level of demethylation of the DNA substrate under conditions sufficient for DNA demethylation, wherein an increase in demethylation of the DNA substrate as compared with the level of demethylation in the absence of the test compound indicates that the test compound is a candidate compound for modulating gene expression in a cell.

70-72. (canceled)


SEQUENCE LISTING (57 sequences)

SEQ ID NO: 1 - DNA, 37 bp, Artificial Sequence
  Synthetic oligonucleotide
acagggaatatatctatatactcccccggaggacctg

SEQ ID NO: 2 - DNA, 37 bp, Artificial Sequence
  Synthetic oligonucleotide
caggtcctccgggggagtatatagatatattccctgt

SEQ ID NO: 3 - DNA, 36 bp, Artificial Sequence
  Synthetic oligonucleotide
aatttcagcatcagttcgccttcatgctgctgatgg

SEQ ID NO: 4 - DNA, 36 bp, Artificial Sequence
  Synthetic oligonucleotide
ccatcagcagcatgaaggcgaactgatgctgaaatt

SEQ ID NO: 5 - protein, 20 aa, Artificial Sequence
  Synthetic peptide
TRGCNLNCGFCSNPASKGRG

SEQ ID NO: 6 - protein, 11 aa, Artificial Sequence
  Synthetic peptide
EWQHRGYGREL

SEQ ID NO: 7 - protein, 26 aa, Artificial Sequence
  Synthetic peptide
PHRCPHIAYTGNICVYCPGGPDSDFE

SEQ ID NO: 8 - protein, 11 aa, Artificial Sequence
  Synthetic peptide
KFQHQGFGTLL

SEQ ID NO: 9 - protein, 26 aa, Artificial Sequence
  Synthetic peptide
PHRCPHISFTGNICIYCPGGPDSDFE

SEQ ID NO: 10 - protein, 11 aa, Artificial Sequence
  Synthetic peptide
KFQHQGFGMLL

SEQ ID NO: 11 - DNA, 21 bp, Artificial Sequence
  Synthetic oligonucleotide
gacugacaggugucgcuuutt

SEQ ID NO: 12 - DNA, 21 bp, Artificial Sequence
  Synthetic oligonucleotide
cauccgaaguuuacacgautt

SEQ ID NO: 13 - DNA, 21 bp, Artificial Sequence
  Synthetic oligonucleotide
guguuuccauaguccgagatt

SEQ ID NO: 14 - DNA, 21 bp, Artificial Sequence
  Synthetic oligonucleotide
gcaccacuacuugaugauatt

SEQ ID NO: 15 - DNA, 21 bp, Artificial Sequence
  Synthetic oligonucleotide
gcuucguaauuacaagauutt

SEQ ID NO: 16 - DNA, 21 bp, Artificial Sequence
  Synthetic oligonucleotide
gaucugcccagaacggauatt

SEQ ID NO: 17 - DNA, 21 bp, Artificial Sequence
  Synthetic oligonucleotide
ccgugguuucuacuaggaatt

SEQ ID NO: 18 - DNA, 21 bp, Artificial Sequence
  Synthetic oligonucleotide
gagcgaauccguaaucauatt

SEQ ID NO: 19 - DNA, 21 bp, Artificial Sequence
  Synthetic oligonucleotide
acccugcgcugaaacccaatt

SEQ ID NO: 20 - DNA, 21 bp, Artificial Sequence
  Synthetic oligonucleotide
gcaguuauucagaauugaatt

SEQ ID NO: 21 - DNA, 20 bp, Artificial Sequence
  Synthetic oligonucleotide
cggctaccacatccaaggaa

SEQ ID NO: 22 - DNA, 18 bp, Artificial Sequence
  Synthetic oligonucleotide
agctggaattaccgcggc

SEQ ID NO: 23 - DNA, 20 bp, Artificial Sequence
  Synthetic oligonucleotide
tgcgagaacgacatcaacat

SEQ ID NO: 24 - DNA, 20 bp, Artificial Sequence
  Synthetic oligonucleotide
tcccggcaaaaacaaataag

SEQ ID NO: 25 - DNA, 20 bp, Artificial Sequence
  Synthetic oligonucleotide
gttctgctgcgacaatgaca

SEQ ID NO: 26 - DNA, 20 bp, Artificial Sequence
  Synthetic oligonucleotide
ttggcttttccaggaatctg

SEQ ID NO: 27 - DNA, 21 bp, Artificial Sequence
  Synthetic oligonucleotide
atgactctggaagaagtccgt

SEQ ID NO: 28 - DNA, 20 bp, Artificial Sequence
  Synthetic oligonucleotide
cagggtccacattcaggact

SEQ ID NO: 29 - DNA, 22 bp, Artificial Sequence
  Synthetic oligonucleotide
gagtcagacctcttctcggaaa

SEQ ID NO: 30 - DNA, 22 bp, Artificial Sequence
  Synthetic oligonucleotide
cgcacctcatcttttagcttct

SEQ ID NO: 31 - DNA, 22 bp, Artificial Sequence
  Synthetic oligonucleotide
ctttcgaaaccaaggatggtag

SEQ ID NO: 32 - DNA, 22 bp, Artificial Sequence
  Synthetic oligonucleotide
cagagaatcatggttttgtcca

SEQ ID NO: 33 - DNA, 22 bp, Artificial Sequence
  Synthetic oligonucleotide
tccgtgctagatatgacccttt

SEQ ID NO: 34 - DNA, 22 bp, Artificial Sequence
  Synthetic oligonucleotide
catcgtgtaaacttcggatgaa

SEQ ID NO: 35 - DNA, 22 bp, Artificial Sequence
  Synthetic oligonucleotide
actccctgcaccactacttgat

SEQ ID NO: 36 - DNA, 22 bp, Artificial Sequence
  Synthetic oligonucleotide
aatccatgccactttgaactct

SEQ ID NO: 37 - DNA, 19 bp, Artificial Sequence
  Synthetic oligonucleotide
ccagtgtccccatgctcaa

SEQ ID NO: 38 - DNA, 19 bp, Artificial Sequence
  Synthetic oligonucleotide
cagctgcatggtccttcca

SEQ ID NO: 39 - DNA, 21 bp, Artificial Sequence
  Synthetic oligonucleotide
cgtactgaaggggaattgtga

SEQ ID NO: 40 - DNA, 21 bp, Artificial Sequence
  Synthetic oligonucleotide
aggaacagctggctttctagg

SEQ ID NO: 41 - DNA, 22 bp, Artificial Sequence
  Synthetic oligonucleotide
gaggaggaggaggaagaagaag

SEQ ID NO: 42 - DNA, 22 bp, Artificial Sequence
  Synthetic oligonucleotide
gctgctttcatctattggctct

SEQ ID NO: 43 - DNA, 20 bp, Artificial Sequence
  Synthetic oligonucleotide
acagaggccttctaagcagc

SEQ ID NO: 44 - DNA, 20 bp, Artificial Sequence
  Synthetic oligonucleotide
ctgaccccaaagagacttcc

SEQ ID NO: 45 - DNA, 24 bp, Artificial Sequence
  Synthetic oligonucleotide
atggggagcagcggtcggtgtagt

SEQ ID NO: 46 - DNA, 24 bp, Artificial Sequence
  Synthetic oligonucleotide
tgcgcgcagcgggaaaagtggtct

SEQ ID NO: 47 - DNA, 22 bp, Artificial Sequence
  Synthetic oligonucleotide
actggagatggcacctaagaaa

SEQ ID NO: 48 - DNA, 22 bp, Artificial Sequence
  Synthetic oligonucleotide
tcgatttctcacctttggtttt

SEQ ID NO: 49 - DNA, 22 bp, Artificial Sequence
  Synthetic oligonucleotide
aaatgacttggagatgcctgat

SEQ ID NO: 50 - DNA, 22 bp, Artificial Sequence
  Synthetic oligonucleotide
tgcgtcttatagagctggtgaa

SEQ ID NO: 51 - DNA, 30 bp, Artificial Sequence
  Synthetic oligonucleotide
gttagagaatttgatagtttttggaatagg

SEQ ID NO: 52 - DNA, 30 bp, Artificial Sequence
  Synthetic oligonucleotide
ccaaaacaaaacctttctcaaacactatat

SEQ ID NO: 53 - DNA, 29 bp, Artificial Sequence
  Synthetic oligonucleotide
taggaaattagtttgaataggtgagaggt

SEQ ID NO: 54 - DNA, 30 bp, Artificial Sequence
  Synthetic oligonucleotide
tcaaacactatattactttaacaattccca

SEQ ID NO: 55 - DNA, 24 bp, Artificial Sequence
  Synthetic oligonucleotide
cttaactacatttcttcttttacc

SEQ ID NO: 56 - DNA, 25 bp, Artificial Sequence
  Synthetic oligonucleotide
agttagygttagtatgtgtatttgt

SEQ ID NO: 57 - DNA, 22 bp, Artificial Sequence
  Synthetic oligonucleotide
tctaaattcctctcttacaact